(19) United States
(12) Patent Application Publication
(10) Pub. No.: US 2024/0331136 A1
(43) Pub. Date: Oct. 3, 2024

(54) MACHINE LEARNING TO PREDICT MEDICAL IMAGE VALIDITY AND TO PREDICT A MEDICAL DIAGNOSIS

(71) Applicant: MDLIVE, Inc., Miramar, FL (US)

(72) Inventor: Nakort E. Valles Leon, Weston, FL (US)

(21) Appl. No.: 18/127,227

(22) Filed: Mar. 28, 2023

(51) Int. Cl. G06T 7/00; G06V 10/70; G16H 20/00; G16H 50/20; G16H 80/00

(52) U.S. Cl. CPC G06T 7/0012; G06V 10/70; G16H 20/00; G16H 50/20; G16H 80/00; G06T 2207/20081; G06T 2207/30168

(57) ABSTRACT

A method performed by a system for providing telehealth services. The method includes receiving inputs from a patient device. The inputs include health data and an image of an ailment. With an image prediction model, the step of determining if the image is of sufficient quality to generate predictions of the ailment. With an ailment prediction model, the method generates one or more predictions of the ailment based on the health data and the image. The method continues with transmitting the predictions and the image to a healthcare provider device. The method proceeds with establishing communication between the patient and healthcare provider devices. The method continues with receiving inputs from the healthcare provider device. The inputs include a confirmation or a rejection of the image and a confirmation or a rejection the predictions. The method proceeds with training the image prediction model and the ailment prediction machine learning model.

### TECHNICAL FIELD

This disclosure relates to systems and methods for using machine learning to predict disease based on images provided by a patient.

### BACKGROUND

Individuals spend a large amount on healthcare every year, and the cost keeps rising. To both combat the rising prices of healthcare and improve patient convenience, some healthcare providers have become increasingly reliant on telehealth or telemedicine services. Such telehealth services allow a patient and a healthcare provider, such as a doctor, to communicate remotely using video and audio communications via separate electronic devices, such as personal computers or smartphones. However, telehealth visits still provide limitations when compared to in-person visits between a patient and a healthcare provider.

### SUMMARY

Objectives of the present disclosure include improving the efficiency of telehealth visits and also improving the accuracy of a healthcare providers' diagnoses that are made during telehealth visits.

One aspect of the present disclosure is related to a system for providing telehealth services to a patient. The system includes a computing device that has at least one processor and at least one memory. The memory includes instructions that, when executed by the at least one processor, cause the at least one processor to receive inputs from a patient device. The inputs include at least health data of the patient and an image of an ailment or a partial image of a patient. With an image prediction machine learning model, the at least one processor determines if the image is of sufficient quality to generate one or more predictions of the ailment. With an ailment prediction machine learning model, the at least one processor generates one or more predictions of the ailment based on the health data of the patient and the image of the ailment. The at least one processor transmits the one or more predictions of the ailment and the image to a healthcare provider device. The at least one processor also establishes communication between the patient device and the healthcare provider device, e.g., through a telehealth system. The at least one processor further receives inputs from the healthcare provider device. The inputs from the healthcare provider device include at least a confirmation or a rejection of the image and including at least one or more predictions of the ailment. The at least one processor trains, based on the inputs from the healthcare provider, the image prediction machine learning model and the ailment prediction machine learning model.

According to another aspect of the present disclosure, the at least one memory further includes instructions that, when executed by the at least one processor, cause the processor to, with the ailment prediction machine learning model, generate one or more predictions of a treatment plan for the patient. The at least one processor also transmits the one or more predictions of the treatment plan for the patient to the healthcare provider device. The inputs from the healthcare provider device include a confirmation or rejection of the one or more predictions of the treatment plan for the patient.

According to yet another aspect of the present disclosure, the system further includes a database that retains images for comparison by the image prediction machine learning model and the ailment prediction machine learning model. In an example embodiment, the retained images are fed back into the prediction engine to refine the models.

According to still another aspect of the present disclosure, the inputs from the healthcare provider device include a confirmation or a rejection of the image. The confirmation of the image or the nonconfirmation of the image can be stored as a digital flag in a record associated with the image.

According to a further aspect of the present disclosure, the at least one memory further includes instructions that, when executed by the at least one processor, cause the processor to, with the image prediction machine learning model, determine if the image of the ailment is a false image that was not taken by the patient. In response to a determination that the image is a false image, the at least one processor requests an additional image from the patient device.

According to yet a further aspect of the present disclosure, the at least one memory further includes instructions that, when executed by the at least one processor, cause the processor to select the healthcare provider device to transmit the one or more predictions of the ailment to among a plurality of healthcare provider devices based on the one or more predictions of the ailment.

According to still a further aspect of the present disclosure, the at least one memory further includes instructions that, when executed by the at least one processor, cause the processor to enhance the image prior to transmitting the image to the healthcare provider device.

According to another aspect of the present disclosure, the computing device is a server that is remote from the patient device and from the healthcare provider device.

According to yet another aspect of the present disclosure, the computing device is associated with the healthcare provider device.

Another aspect of the present disclosure is related to a method performed by a system for providing telehealth services to a patient. The system comprises a computing device with at least one processor and at least one memory. The method comprises the step of receiving inputs from a patient device. The inputs include at least health data of the patient and an image of an ailment. With an image prediction machine learning model, the method continues with the step of determining if the image is of sufficient quality to generate one or more predictions of the ailment. With an ailment prediction machine learning model, the method proceeds with the step of generating one or more predictions of the ailment based on the health data of the patient and the image of the ailment. The method continues with the step of transmitting the one or more predictions of the ailment and the image to a healthcare provider device. The method proceeds with the step of establishing communication between the patient device and the healthcare provider device. The method continues with the step of receiving inputs from the healthcare provider device. The inputs from the healthcare provider device include at least a confirmation or a rejection of the image and including a confirmation or a rejection of the one or more predictions of the ailment. The method proceeds with the step of training, based on the inputs from the healthcare provider, the image prediction machine learning model and the ailment prediction machine learning model.

According to another aspect of the present disclosure, the method further includes the step of, with the ailment prediction machine learning model, generating one or more predictions of a treatment plan for the patient. The method continues with the step of transmitting the one or more predictions of the treatment plan for the patient to the healthcare provider device. The inputs from the healthcare provider device include a confirmation or rejection of the one or more predictions of the treatment plan for the patient.

According to yet another aspect of the present disclosure, the method further includes the step of storing the image in a database for access by the image prediction machine learning model and the ailment prediction machine learning model.

According to still another aspect of the present disclosure, the inputs from the healthcare provider device include a confirmation or a rejection of the image.

According to a further aspect of the present disclosure, with the image prediction machine learning model, the method further includes the step of determining if the image of the ailment is a false image that was not taken by the patient. In response to a determination that the image is a false image, the method continues with the step of requesting an additional image from the patient device.

According to yet a further aspect of the present disclosure, the method further includes the step of selecting the healthcare provider device to transmit the one or more predictions of the ailment to among a plurality of healthcare provider devices based on the one or more predictions of the ailment.

According to still a further aspect of the present disclosure, the method further includes the step of enhancing the image prior to transmitting the image to the healthcare provider device.

Yet another aspect of the present disclosure is related to a system for providing telehealth services to a patient. The system includes a cloud computing device with at least one processor and at least one memory. The memory including instructions that, when executed by the processor, cause the processor to receive inputs from a patient device, the inputs including at least health data of the patient and an image of an ailment. With an image prediction machine learning model, the processor determines if the image is of sufficient quality to generate one or more predictions of the ailment. With an ailment prediction machine learning model, the processor generates one or more predictions of the ailment and one or more predictions of a treatment plan based on the health data of the patient and the image of the ailment. The processor selects a healthcare provider device of a plurality of healthcare provider devices based on the one or more predictions of the ailment. The processor transmit the one or more predictions of the ailment and the one or more predictions of the treatment plan and the image to the healthcare provider device. The processor establishes communication between the patient device and the healthcare provider device. The processor receives inputs from the healthcare provider device. The inputs from the healthcare provider device include at least a confirmation or rejection of the image, a confirmation or a rejection of the one or more predictions of the ailment, and a confirmation or rejection of the one or more predictions of the treatment plan. The processor trains, based on the inputs from the healthcare provider, the image prediction machine learning model and the ailment prediction machine learning model.

According to another aspect of the present disclosure, the at least one memory further includes instructions that, when executed by the at least one processor, cause the processor to request an additional image from the patient device in response to a determination that the image of the ailment is not of sufficient quality to generate the one or more predictions of the ailment.

According to yet another aspect of the present disclosure, the inputs from the healthcare provider device include a confirmation or a rejection of the image.

According to still another aspect of the present disclosure, the at least one memory further includes instructions that, when executed by the at least one processor, cause the processor to determine if the image of the ailment is a false image that was not taken by the patient with the image prediction machine learning model. In response to a determination that the image is a false image, the instructions cause the processor to request an additional image from the patient device.

### BRIEF DESCRIPTION OF THE DRAWINGS

The disclosure is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity.

FIG. 1A is a block diagram of an example image verification and prediction system;

FIG. 1B is a block diagram of an example computing device of the image verification and prediction system of FIG. 1A;

FIG. 2 depicts a flow chart that illustrates the steps of using machine learning models to determine if an image uploaded by a patient is a false image and then using machine learning models to identify an ailment according to one embodiment of the present disclosure;

FIG. 3 depicts a flow chart that illustrates the steps of using machine learning to predict the identity of an ailment according to another embodiment of the present disclosure;

FIG. 4 depicts a flow chart that illustrates the steps of using machine learning to determine if an image is of sufficient clarity for a prediction and then to make a prediction of an ailment;

FIG. 5 is a block diagram of an example image validation platform, according to some embodiments;

FIG. 6 is an example database that may be deployed within the system of FIG. 1, according to some embodiments;

FIG. 7 is a block diagram illustrating an example software architecture, which may be used in conjunction with various hardware architectures herein described;

FIG. 8 is a block diagram illustrating components of a machine, according to some example embodiments;

FIG. 9 is a block diagram of an example image validation system that may be deployed within the various systems described herein, according to some embodiments;

FIG. 10 is a functional block diagram of an example neural network that can be used for the inference engine or other functions (e.g., engines) as described herein to produce a predictive model; and

FIG. 11 illustrates a machine learning engine for generating a predictive model using images to approve of an image or suggest a determining feedback in accordance with at least one example of this disclosure.

### DETAILED DESCRIPTION

The following discussion is directed to various embodiments of the invention. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and not intended to intimate that the scope of the disclosure, including the claims, is limited to that embodiment.

The present disclosure allows for both improved efficiency of telehealth visits and allows for improved diagnoses by a healthcare provider, such as a doctor, a nurse, a pharmacist, or a medical assistant. As discussed in further detail below, these results are realized by way of a telehealth system which allows a user to upload one or more images to a computing device. Using machine learning (ML) and artificial intelligence, the computing device automatically processes and analyzes the images. The computing device automatically determines if the images are of sufficient quality to establish an accurate diagnosis. The computing device can also provide a diagnosis recommendation or prediction and treatment regimen to the healthcare provider. The healthcare provider can then choose to relay the diagnosis recommendation and treatment regimen to the patient or to offer the patient a different diagnosis, e.g., through the telehealth system. If the healthcare provider offers the patient a different diagnosis than the one that was recommended by the computing device, then the ML models of the computing device use this discrepancy to improve future diagnoses by modifying the model.

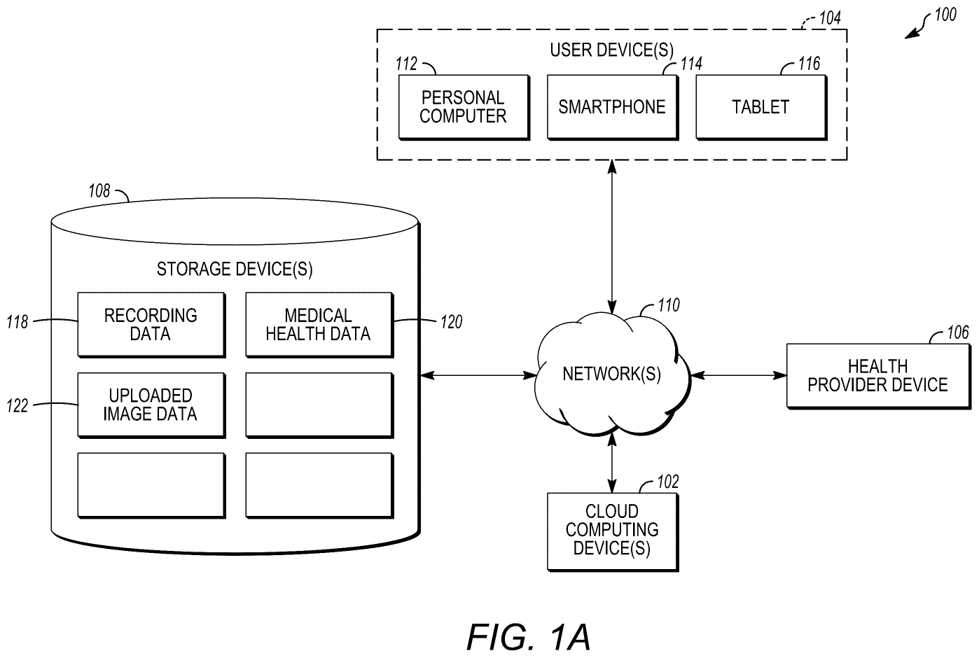

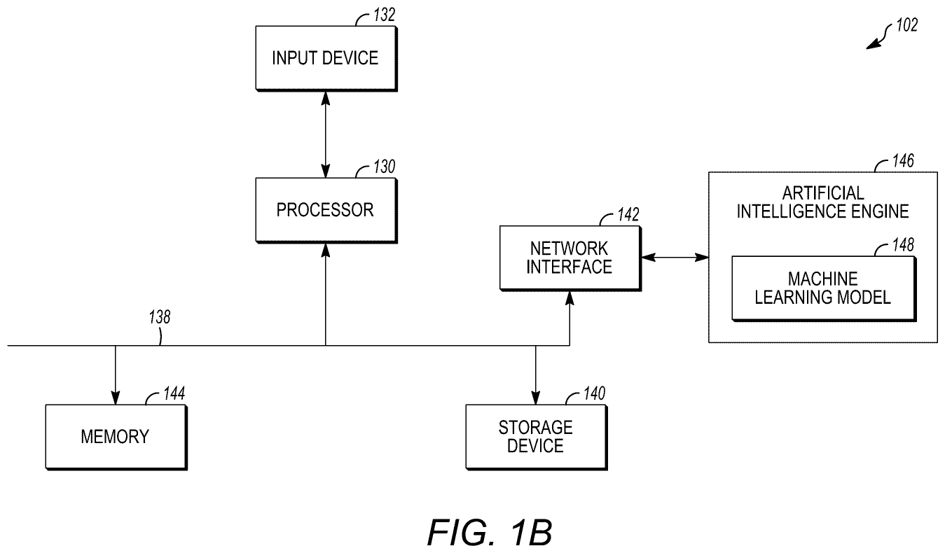

FIG. 1A is a block diagram of an example implementation of a system 100 for providing telehealth services to a patient. The system 100 includes a computing device 102, a user device 104 (patient device), a healthcare provider device 106, and a storage device 108 that are all in communication with one another via a network 110. The user device 104 can be a personal computer 112, a smartphone 114, a tablet 116, or any suitable type of device that can communicate via the network 110 and has a camera, a microphone, and a speaker for allowing the patient to communicate with the healthcare provider.

Examples of the network 110 include a Global System for Mobile Communications (GSM) network, a code division multiple access (CDMA) network, 3rd Generation Partnership Project (3GPP), an Internet Protocol (IP) network, a Wireless Application Protocol (WAP) network, or an IEEE 802.11 standards network, as well as various combinations of the above networks. The network 110 may include an optical network. The network 110 may be a local area network or a global communication network, such as the Internet. Moreover, although the system 100 shows a single network 110, multiple networks can be used. The multiple networks may communicate in series and/or parallel with each other to link the devices 102-108.

The healthcare provider device 106 may be associated with a doctor, a nurse, a pharmacist, a medical assistant, or any suitable type of healthcare provider.

The storage device 108 may include non-transitory storage (for example, solid-state memory, hard disk, CD-ROM, etc.) and is in communication with the computing device 102, the user device 104, and the healthcare provider device 106 over the network 110. The non-transitory storage may provide short term or long term storage of recording data 118, medical health data 120, uploaded images data 122, and any suitable data pertaining to a telehealth appointment between the user on the user device 104 and a healthcare provider on the healthcare provider device 106.

Turning now to FIG. 1B, the computing device 102 may include any suitable computing device, such as a mobile computing device, a desktop computing device, a laptop computing device, a server computing device, other suitable computing device, or a combination thereof. During a telehealth appointment between a patient using the user device 104 and a healthcare provider using the healthcare provider device 106, the computing device 102 is in communication with both of these devices 104, 106 and can receive and process data from each.

The computing device 102 may include a processor 130 configured to control the overall operation of computing device 102. The processor 130 may include any suitable processor, such as those described herein. The computing device 102 may also include a user input device 132 that is configured to receive input from the user device 104 and to communicate signals representing the input received from the user to the processor 130.

A data bus 138 may be configured to facilitate data transfer between, at least, a storage device 140 and the processor 130. The computing device 102 may also include a network interface 142 configured to couple or connect the computing device 102 to various other computing devices or network devices via a network connection, such as a wired or wireless connection, such as the network 110. In some embodiments, the network interface 142 includes a wireless transceiver.

The storage device 140 may include a single disk or a plurality of disks (e.g., hard drives), one or more solid-state drives, one or more hybrid hard drives, and the like. The storage device 140 may include a storage management module that manages one or more partitions within the storage device 140. In some embodiments, storage device 140 may include flash memory, semiconductor (solid state) memory or the like. The computing device 108 may also include a memory 144. The memory 144 may include Random Access Memory (RAM), a Read-Only Memory (ROM), or a combination thereof. The memory 144 may store programs, utilities, or processes to be executed in by the processor 130. The memory 144 may provide volatile and/or non-volatile data storage, and stores instructions related to the operation of the computing device 108.

In some embodiments, the processor 130 may be configured to execute instructions stored on the memory 144 to, at least, perform the systems and methods described herein. For example, the processor 130 may be configured to receive a request for coverage of a service received by a member under a health insurance plan, determine eligibility of the member under the health insurance plan, and determine if the service is covered by the health insurance plan. The processor 130 may be further configured to determine a first healthcare attribute and a second healthcare attribute associated with the service and generate a response to the request for coverage of the service under the health insurance plan indicating a value associated with the second healthcare attribute. The response may be generated in response to a value associated with the first healthcare attribute being greater than a value associated with the second healthcare attribute. As used herein, healthcare attributes may refer to any attributes associated with a requested healthcare service. For example, the healthcare attributes may include patient information (e.g., patient's name, gender, etc.), information related to the service (e.g., diagnosis code, place of service, date of service, service code, etc.), and health insurance information (e.g., subscriber identification number or plan number).

The computing device 102 further includes an artificial intelligence engine 146 that includes a ML model 148 that is capable of processing images submitted by the user via the user device 104, compare those images to other images in an image database, and predict a diagnosis and treatment plan based on those images. Beyond only the images, some healthcare attributes that may be processed by the ML model 148 when making a diagnosis and treatment plan prediction include patient information (e.g., gender, occupation, age, location, etc.) and a patient's medical history including surgical, dental, x-ray, pharmaceutical, travel history, and other suitable records.

Additionally, or alternatively, the ML model generator may also include a ML application that implements the ML algorithm to the healthcare attribute association model. When the ML algorithm is implemented, it may find patterns between the healthcare attributes to map the healthcare attributes to each other and outputs a model that captures the associations between healthcare attributes. The healthcare attributes model may be generated using any suitable techniques, including supervised ML model generation algorithms such as supervised vector machines (SVM), linear regression, logistic regression, naïve Bayes, linear discriminant analysis, decision trees, k-nearest neighbor algorithm, neural networks, recurrent neural network, etc. In some embodiments, unsupervised learning algorithms may be used such as clustering or neural networks. In some embodiments, the ML model generator may implement a gradient boosted tree algorithm or other decision tree algorithm to generate and/or train the healthcare attributes model in the form of a decision tree. The ML model generator may implement an artificial neural network learning algorithm to generate the diagnosis prediction, such as a neural network that is an interconnected group of artificial neurons. The neural network may be presented with information related to the request for a healthcare service to identify healthcare attributes associated with the requested healthcare service.

In some embodiments, the ML model generator may be configured to generate an attribute association model that generates one or more predictions indicating associations between attributes or healthcare attributes. For example, the model generator includes a ML algorithm. The ML algorithm may be provided with attributes associated with previous requests for services as input and is executed by model generator to generate the attribute association model. The attribute association model may generate predictions indicating associations between attributes associated with data processing services. Some attributes may include member information (e.g., gender, occupation, age, location, etc.), information related to the service (e.g., diagnosis code, place of service, date of service, service code, etc.), and participation information (e.g., subscriber identification number or plan number, deductible, insurance type, participation level, etc.).

Additionally, or alternatively, ML model generator may also include a ML application that implements the ML algorithm to the attribute association model. When the ML algorithm is implemented, it may find patterns between the attributes to map the attributes to each other, and output a model that captures the associations between attributes. The attributes model may be generated using any suitable techniques, including supervised ML model generation algorithms such as supervised vector machines (SVM), linear regression, logistic regression, naïve Bayes, linear discriminant analysis, decision trees, k-nearest neighbor algorithm, neural networks, recurrent neural network, etc. In some embodiments, unsupervised learning algorithms may be used such as clustering or neural networks. Note that the attributes model may be generated in various forms. In accordance with some embodiments, the attributes model may be generated according to a suitable machine-learning algorithm mentioned elsewhere herein or otherwise known. In some embodiments, the ML model generator may implement a gradient boosted tree algorithm or other decision tree algorithm to generate and/or train the attributes model in the form of a decision tree. The decision tree may be traversed with input data (information related to the request for a service, etc.) to identify one or more attributes associated with the service and data processing request. Alternatively, the ML model generator may implement an artificial neural network learning algorithm to generate the attributes model as a neural network that is an interconnected group of artificial neurons. The neural network may be presented with information related to the request for a healthcare service to identify healthcare attributes associated with the requested data processing service.

The present system can additionally connect a patient device with a provider device to provide a telehealth visit. Examples of telehealth visits are described in U.S. patent application Ser. No. 17/748,170 (titled TELEHEALTH CONTROL SYSTEM AND METHOD) and Ser. No. 17/486,251 (titled TELEHEALTH PROVIDER MANAGEMENT SYSTEM AND METHOD), which are hereby incorporated by reference. By way of example, a telehealth system may be used to establish and conduct remote encounters between healthcare providers and patients. A remote encounter may include an interaction (e.g., meeting, medical appointment, medical consultation, etc.) between a healthcare provider and a patient via video conference or teleconference while the healthcare provider and the patient are in different locations (e.g., different rooms, different buildings, different towns or cities, different zip codes, different states, or different countries). The healthcare provider can be a Doctor of Medicine, Doctor of Osteopathy, a podiatrist, a dentist, a chiropractor, a clinical psychologist, an optometrist, a nurse practitioner, a nurse-midwife, or a clinical social worker. The telehealth system can establish remote encounters between healthcare providers and patients, not all embodiments of the inventive subject matter are limited to telehealth systems. For example, the telehealth system optionally can be used to establish and conduct remote encounters between real estate agents and clients, between attorneys and clients, courts and parties to a civil lawsuit or criminal court action, teachers and students, governmental meetings, voter and election systems, or any other service provider and customer who are connected via an electronic communication system.

The telehealth system includes a telehealth control system, which can be connected to the data bus 138 or the network 110, can include hardware circuitry having and/or connected with one or more processors (e.g., microprocessors, field programmable gate arrays, integrated circuits, etc.) that perform the operations described in connection with the telehealth control system. The control system communicates (wirelessly and/or via wired connections) with provider computing devices 106 and consumer or patient computing devices 104. The computing devices 104, 106 can represent laptop computers, desktop computers, tablet computers, or the like, and the computing devices 104, 106 can represent mobile phones.

The telehealth control system can manage a virtual encounter between the providers and patients by establishing communication channels between a computing device 106 of the provider(s) and a computing device 104 of the patient. This communication channel can be a videoconference or a teleconference that extends through one or more computer communication networks, such as the Internet, one or more intranets, one or more local area networks, or the like. The computing devices 104, 106 may have software applications installed or otherwise running thereon to establish a secure connection between (a) the provider computing device 106 and (b) the patient computing device 104. These software applications can be commercial or proprietary applications used by a company or government to manage the remote encounters between providers and patients, e.g., in an encrypted or secure manner. The applications can be installed in internal computer memories of the computing devices 104, 106 or may be accessed via web pages provided by a telehealth server. One example of such a software application or service is MDLIVE healthcare services that provides remote healthcare services e.g., via telephone, video, email, mobile devices or a global computer network.

The secure connection can extend through the network(s) 110 and be encrypted or otherwise protected from outside parties to ensure confidentiality of the information communicated between the provider and the patient. For example, the videoconference or teleconference channel can extend through one or more digital subscriber lines, cable modems, network fibers, wireless networks, satellite networks, broadband over powerline connections, etc., using the transmission control protocol over Internet protocol, or another protocol.

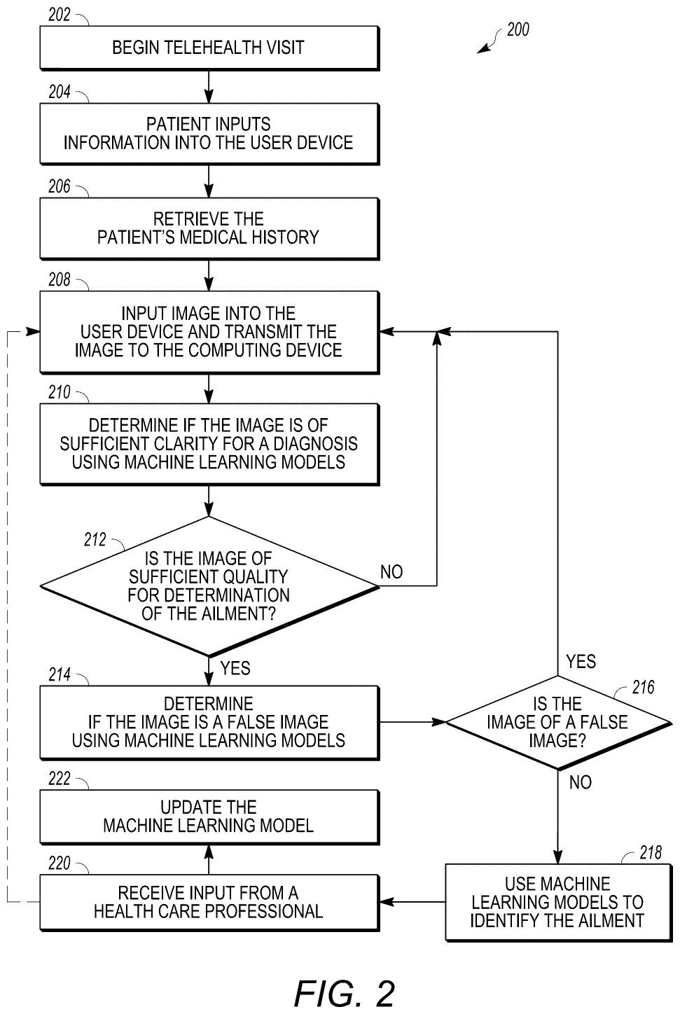

To explore this further, FIG. 2 will now be described. FIG. 2 shows an exemplary embodiment of a method 200 for processing an image uploaded by a patient to the computing device. The method 200 is performed by processing logic that may include hardware (circuitry, dedicated logic, etc.), software (such as is run on a general purpose computer system or a dedicated machine), or a combination of both. The method 200 and/or each of their individual functions, routines, subroutines, or operations may be performed by one or more processors of a computing device (e.g., any component of FIG. 1, such as cloud computing device 102 or the healthcare provider device 106). In certain implementations, the method 200 may be performed by a single processing thread. Alternatively, the method 200 may be performed by two or more processing threads, each thread implementing one or more individual functions, routines, subroutines, or operations of the methods.

For simplicity of explanation, the method 200 is depicted and described as a series of operations. However, operations in accordance with this disclosure can occur in various orders and/or concurrently, and with other operations not presented and described herein. For example, the operations depicted in the method 200 may occur in combination with any other operation of any other method disclosed herein. Additionally, or alternatively, not all illustrated operations may be required to implement the method 200 in accordance with the disclosed subject matter. In addition, those skilled in the art will understand and appreciate that the method 200 could alternatively be represented as a series of interrelated states via a state diagram or events.

At step 202, a telehealth visit begins with the establishment of electrical communication between the user device 104 and the computing device. At step 204, the patient inputs information into the user device 104 related to their health and current physical status. This information may include, for example: name, location, biological sex, recent travel history, blood pressure, medications currently being taken, height, weight, symptoms, etc. The information that is input by the patient is then automatically transmitted from the user device 104 to the computing device via the network 110.

At step 206, the computing device retrieves a medical history of the patient. The medical history may include, for example: medication history, previous diagnosis, surgical history, drug use history, etc. The medical history can be retrieved from external servers, such as the storage device 108 and/or data servers maintained by hospital or medical care systems.

At step 208, the patient inputs an image of a physical ailment into the user device 104, and the user device 104 automatically transmits the image to the computing device. As used herein, the physical ailment could be any type of condition or symptom of a potential disease or injury that manifests in such a way that it can appear in a photograph, e.g., a rash, a cut, a boil, a deformity, discolored urine, discolored stool, an area of discolored skin, etc. The image may be generated by the patient using a camera built into the user device 104 to take a picture of the physical ailment. In some cases, an app on the user device 104 can be utilized to instruct the user how to optimize image quality. For example, software in the user device 104 can allow the user device 104 to analyze the camera in the user device and instruct the user to, for example, move closer or further away from the physical ailment. The software in the user device 104 can also adjust camera settings (for example, flash on or off, focus, brightness, contrast, resolution, exposure time, etc.) to optimize image quality before the image is even captured by the user device 104.

If the user device does not have a camera built into it, then the computing device can send a link to a second device that the user can access which allows the user to use the second device so that the second device can be used to generate the image. The user can also input an image that was previously generated using any device and is currently stored in a memory of the user device 104.

At step 210, using artificial intelligence and ML models, the computing device processes the image and uses artificial intelligence and ML models to determine if the image is of sufficient quality for a diagnosis. At this step, the computing device may analyze the clarity, brightness, resolution, etc. of the image and analyze the focus of the image on the portion which contains the physical ailment. That is, if the background of the image is clear but the physical ailment is blurry, then the computing device can reject the image.

At decision step 212, it is determined if the image is of sufficient quality for the machine models to make a determination of the ailment. If the answer at decision step 212 is “no” (the computing device rejects the image), then the process returns to step 208 to allow the user to input another image. In some embodiments, an app on the user device may allow the patient to force an image through decision step 212 if the patient does not believe it possible to capture a better quality image.

If the answer at decision step 212 is “yes,” then at step 214, using artificial intelligence and ML models, the computing device processes the image to determine if the image is a false image. This may involve a comparison of the image to a database of stored images to determine if the image is a duplicate of another image. It may also involve analyzing metadata connected with the image and looking for inconsistencies, e.g., an image generation date that is significantly before the uploading of the image or an image location that is not consistent with a current location of the patient.

At decision step 216, it is determined if the image is a false image. If the answer at decision step 216 is “yes,” then the process returns to step 208.

If the answer at decision step 216 is “no,” then at step 218, using artificial intelligence and ML models, the computing system generates at least one prediction of the ailment contained in the image by comparing the image to a database of other images and associated ailments connected with those images and also by comparing the patient information data received in steps 204 and 206 to other patient data and the positive verifications of the ailments of the other patients.

At step 220, the computing system transmits the patient's information, the image, and the prediction of the ailment to the healthcare provider device 106, and a healthcare professional makes a selection of if the computing device correctly identified the ailment. The healthcare professional can also reject the image and send the user back to step 208.

If the healthcare professional determines that the computing device did not correctly predict the ailment contained in the image, then the healthcare professional inputs into the healthcare provider device 106 what the ailment is, i.e., the correct diagnosis. Regardless of whether the computing device correctly predicted the ailment or not, at step 222, the computing device updates the ML model by inputting the image and the correct identification of the ailment into a database for consideration in future telehealth visits with both the same patient and other patients who may have the same or a similar ailment to improve future predictions by the artificial intelligence using the ML models. If any images were rejected by the healthcare provider as being of insufficient quality to accurately predict the ailment, the computing device can also add those rejected images to the database to further train the ML models to more accurately predict if an image is of sufficient quality in future telehealth visits with the same and other patients.

In an example embodiment, the step 212 of determination if an image of sufficient quality to assist a healthcare provider in diagnosing a patient can be performed while the patient is waiting to be connected to the healthcare provider device. Thus, when the connection occurs the provider device has already displayed an approved, validated image of the patient illness. In an example, the provider, through the related device, can reject the image and the patient remains in the waiting room until an adequate, approved image is uploaded. The provider is then free to engage a different patient, through their related device. The healthcare system can flag a patient as not available for connecting a patient to a provider until an approved image is uploaded and approved.

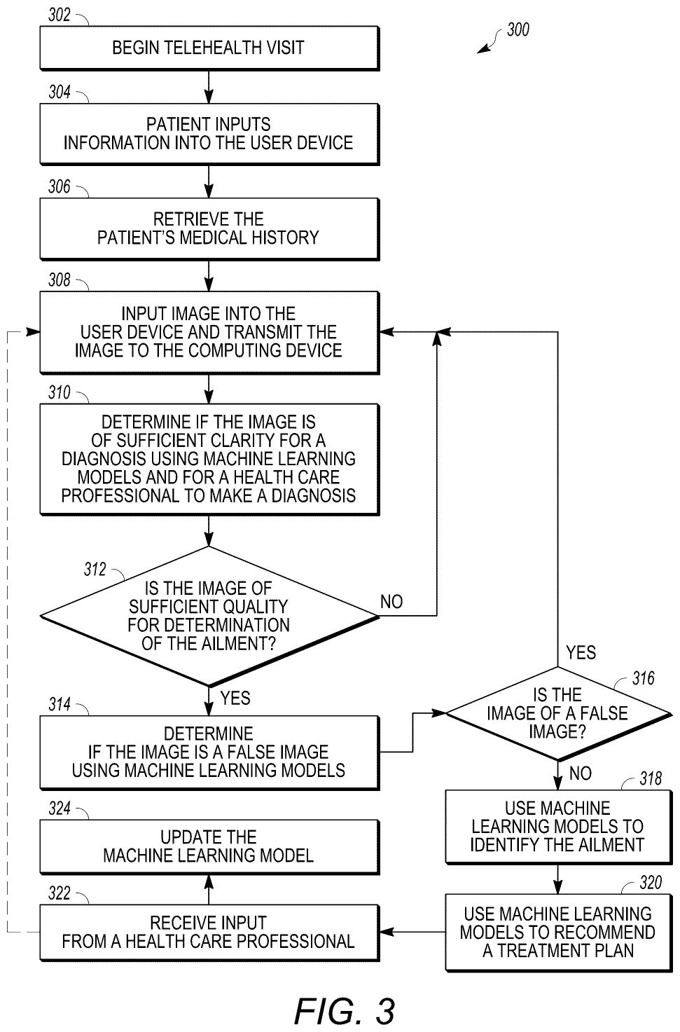

Turning now to FIG. 3, a second embodiment of the method 300 is depicted. At step 302, a telehealth visit begins with the establishment of electrical communication between the user device 104 and the computing device. At step 304, the patient inputs information into the user device 104 related to their health and current physical status. This information may include, for example: name, location, biological sex, recent travel history, blood pressure, medications currently being taken, height, weight, symptoms, etc. The information that is input by the patient is then automatically transmitted from the user device 104 to the computing device via the network 110.

At step 306, the computing device retrieves a medical history of the patient. The medical history may include, for example: medication history, previous diagnosis, surgical history, drug use history, etc. The medical history can be retrieved from external servers, such as the storage device 108 and/or data servers maintained by hospital or medical care systems.

At step 308, the patient inputs an image of a physical ailment into the user device 104, and the user device 104 automatically transmits the image to the computing device. As used herein, the physical ailment could be any type of condition or symptom of a potential disease or injury that manifests in such a way that it can appear in a photograph, e.g., a rash, a cut, a boil, a deformity, discolored urine, discolored stool, an area of discolored skin, etc. The image may be generated by the patient using a camera built into the user device 104 to take a picture of the physical ailment. In some cases, an app on the user device 104 can be utilized to instruct the user how to optimize image quality. For example, software in the user device 104 can allow the user device 104 to analyze the camera in the user device and instruct the user to, for example, move closer or further away from the physical ailment. The software in the user device 104 can also adjust camera settings (for example, flash on or off, focus, brightness, contrast, resolution, exposure time, etc.) to optimize image quality before the image is even captured by the user device 104.

If the user device does not have a camera built into it, then the computing device can send a link to a second device that the user can access which allows the user to use the second device so that the second device can be used to generate the image. The user can also input an image that was previously generated using any device and is currently stored in a memory of the user device 104.

At step 310, using artificial intelligence and ML models, the computing device processes the image and uses artificial intelligence and ML models to determine if the image is of sufficient quality for a diagnosis. Specifically, the computing device automatically determines if the image is of sufficient quality for both itself and a healthcare professional to make a diagnosis. At this step, the computing device may analyze the clarity, brightness, resolution, etc. of the image and analyze the focus of the image on the portion which contains the physical ailment. That is, if the background of the image is clear but the physical ailment is blurry, then the computing device can reject the image.

At decision step 312, it is determined if the image is of sufficient quality for the machine models and for the healthcare professional to make a determination of the ailment. If the answer at decision step 312 is “no” (the computing device rejects the image), then the process returns to step 308 to allow the user to input another image. In some embodiments, an app on the user device may allow the patient to force an image through decision step 312 if the patient does not believe it possible to capture a better quality image.

If the answer at decision step 312 is “yes,” then at step 314, using artificial intelligence and ML models, the computing device processes the image to determine if the image is a false image. This may involve a comparison of the image to a database of stored images to determine if the image is a duplicate of another image and may include an analysis of metadata associated with the image.

At decision step 316, it is determined if the image is a false image. If the answer at decision step 316 is “yes,” then the process returns to step 308.

If the answer at decision step 316 is “no,” then at step 318, using artificial intelligence and ML models, the computing system generates at least one prediction of the ailment contained in the image by comparing the image to a database of other images and associated ailments connected with those images and also using the patient information data received in steps 304 and 306.

At step 320, using artificial intelligence and ML models, the computing device generates at least one recommended treatment plan for the patient. The computing device uses the data from the image and the patient's health history and information to establish the at least one recommended treatment. The recommended treatment plan could include, for example, a medication regimen, physical therapy, a lifestyle change, etc.

At step 322, the computing system transmits the patient's information, the image, the prediction of the ailment, and the predicted treatment plan to the healthcare provider device 106. Using the healthcare provider device 106, the healthcare professional then makes a selection of if the computing device correctly identified the ailment and the recommended treatment or not, i.e., if the predicted diagnoses was accurate. If the healthcare professional determines that the computing device did not correctly identify the ailment contained in the image and/or the computing device did not correctly identify a proper recommendation, then the healthcare professional inputs into the healthcare provider device 106 what the correct ailment and/or treatment is. If the healthcare professional does not believe the image to be of sufficient quality to make a correct diagnosis, then the healthcare professional can send the user back to step 308 to input a new image.

Regardless of whether the computing device correctly predicted the ailment or not, at step 324, the computing device updates the ML model by inputting the image, the correct identification of the ailment, and the correct treatment into a database for consideration in future telehealth visits with both the patient and other patients. If any images were rejected, the computing device can also add those rejected images to the database to further train the ML models and allow the artificial intelligence to more accurately predict if an image is of sufficient quality during future telehealth visits with the patient or other patients.

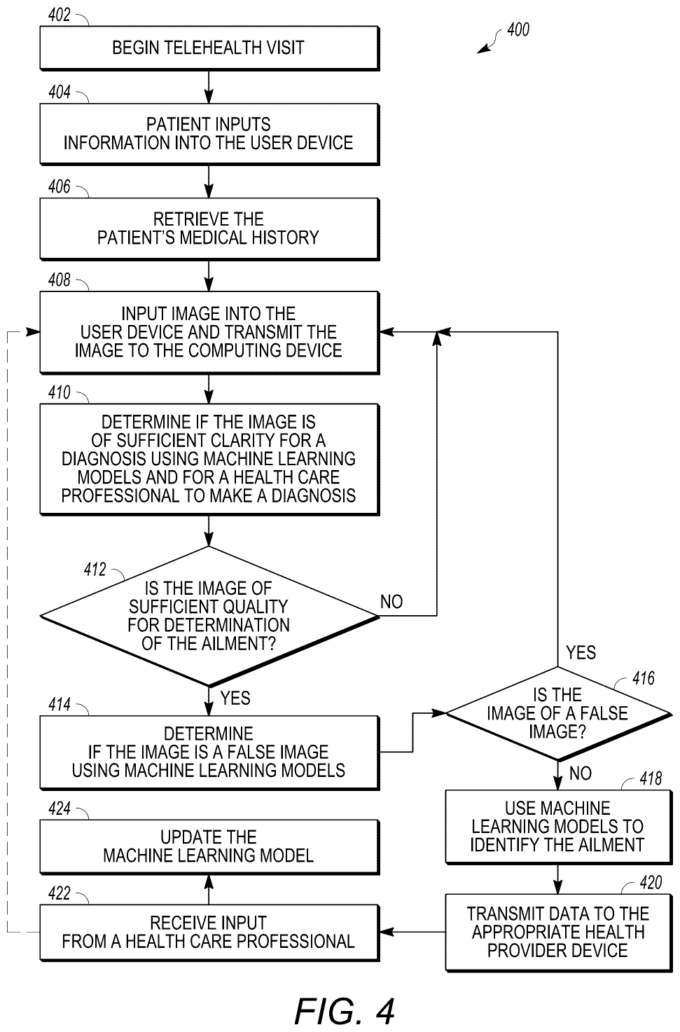

Turning now to FIG. 4, a third embodiment of the method 400 is depicted. At step 402, a telehealth visit begins with the establishment of electrical communication between the user device 104 and the computing device. At step 404, the patient inputs information into the user device 104 related to their health and current physical status. This information may include, for example: name, location, biological sex, recent travel history, blood pressure, medications currently being taken, height, weight, symptoms, etc. The information that is input by the patient is then automatically transmitted from the user device 104 to the computing device via the network 110.

At step 406, the computing device retrieves a medical history of the patient. The medical history may include, for example: medication history, previous diagnosis, surgical history, drug use history, etc. The medical history can be retrieved from external servers, such as the storage device 108 and/or data servers maintained by hospital or medical care systems.

At step 408, the patient inputs an image of a physical ailment into the user device 104, and the user device 104 automatically transmits the image to the computing device. As used herein, the physical ailment could be any type of condition or symptom of a potential disease or injury that manifests in such a way that it can appear in a photograph, e.g., a rash, a cut, a boil, a deformity, discolored urine, discolored stool, an area of discolored skin, etc. The image may be generated by the patient using a camera built into the user device 104 to take a picture of the physical ailment. In some cases, an app on the user device 104 can be utilized to instruct the user how to optimize image quality. For example, software in the user device 104 can allow the user device 104 to analyze the camera in the user device and instruct the user to, for example, move closer or further away from the physical ailment. The software in the user device 104 can also adjust camera settings (for example, flash on or off, focus, brightness, contrast, resolution, exposure time, etc.) to optimize image quality before the image is even captured by the user device 104.

If the user device does not have a camera built into it, then the computing device can send a link to a second device that the user can access which allows the user to use the second device so that the second device can be used to generate the image. The user can also input an image that was previously generated using any device and is currently stored in a memory of the user device 104.

At step 410, using artificial intelligence and ML models, the computing device processes the image and uses artificial intelligence and ML models to determine if the image is of sufficient quality for a diagnosis. Specifically, the computing device automatically determines if the image is of sufficient quality for both itself and a healthcare professional to make a diagnosis. At this step, the computing device may analyze the clarity, brightness, resolution, etc. of the image and analyze the focus of the image on the portion which contains the physical ailment. That is, if the background of the image is clear but the physical ailment is blurry, then the computing device can reject the image.

At decision step 312, it is determined if the image is of sufficient quality for the machine models and for the healthcare professional to make a determination of the ailment. If the answer at decision step 412 is “no” (the computing device rejects the image), then the process returns to step 408 to allow the user to input another image. In some embodiments, an app on the user device may allow the patient to force an image through decision step 412 if the patient does not believe it possible to capture a better quality image.

If the answer at decision step 412 is “yes,” then at step 414, using artificial intelligence and ML models, the computing device processes the image to determine if the image is a false image. This may involve a comparison of the image to a database of stored images to determine if the image is a duplicate of another image.

At decision step 416, it is determined if the image is a false image. If the answer at decision step 416 is “yes,” then the process returns to step 408.

If the answer at decision step 416 is “no,” then at step 418, using artificial intelligence and ML models, the computing system generates at least one prediction of the ailment contained in the image by comparing the image to a database of other images and associated ailments connected with those images and also using the patient information data received in steps 404 and 406.

At step 420, using artificial intelligence and ML models, the computing device predicts at least one treatment for the patient. The computing device uses the data from the image and the patient's health history and information to establish the predicted treatment plan. The predicted treatment plan could include, for example, a medication regimen; physical therapy; a lifestyle change; one or more follow-up tests; etc. The computing device then monitors a plurality of healthcare provider devices 106 associated with a plurality of different available healthcare professionals. These healthcare professionals may have differing specialties, e.g., one could be a dermatologist and another could be an oncologist. If the computing device predicts that the ailment is eczema, then then the computing device can establish a connection with the healthcare provider device 106 associated with the available dermatologist. On the other hand, if the computing device predicts that the ailment is melanoma, then the computing device can establish a connection with the healthcare provider device 106 associated with the available oncologist.

At step 422, the computing system transmits the patient's information, the image, the at least one prediction of the ailment, and the at least one predicted treatment plan to the healthcare provider device 106. Using the healthcare provider device 106, the healthcare professional then makes a selection of if the computing device correctly predicted the ailment and the recommended treatment or not. If the healthcare professional determines that the computing device did not correctly identify the ailment contained in the image and/or the computing device did not correctly identify a proper recommendation, then the healthcare professional inputs into the healthcare provider device 106 what the correct ailment and/or treatment is. If the healthcare professional does not believe the image to be of sufficient quality to make a correct diagnosis, then the healthcare professional can send the user back to step 408 to input a new image.

Regardless of whether the computing device correctly identified the ailment or not, at step 424, the computing device updates the ML model by inputting the image, the correct identification of the ailment, and the correct treatment plan into a database for consideration in future telehealth visits with both the patient and other patients. If any images were rejected, the computing device can also add those rejected images to the database to further train the ML models to more accurately predict if an image is of sufficient quality during future telehealth visits with the patient and other patients. If the computing device identified the incorrect healthcare professional to send the data to, then this information can also be added to the data base to further improve the ML models for future telehealth visits.

In some embodiments, the computing device may also use ML models and artificial intelligence to enhance the image that is uploaded by the patient to allow for an improved identification of the ailment. For example, the computing device may brighten an image of a rash on a person who has a dark skin tone to make the rash more visible for both the artificial intelligence and for the healthcare professional to identify the ailment and recommend a treatment.

In some embodiments, the “image” could be a video that is transmitted to the computing device. The video could also include, in addition to the image component, an audio component.

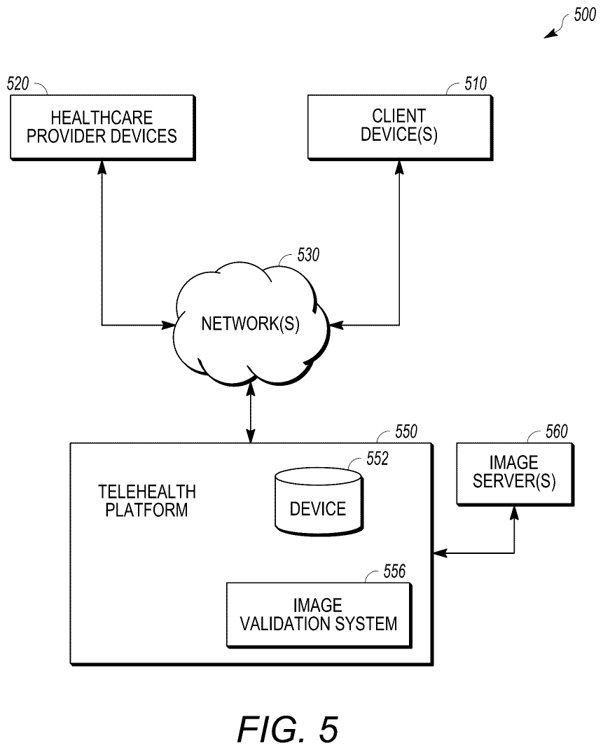

FIG. 5 is a block diagram showing an example telehealth system 500 according to various exemplary embodiments. The telehealth system 500 includes one or more client devices 510, one or more healthcare provider devices 520, an telehealth platform 550, and one or more image servers 560 that are communicatively coupled over a communication network 530 (e.g., intranet, global computer network, Internet, telephony network or the like). Each of the one or more image servers 560 hosts an application that enables the telehealth platform 550 to analyze images contained on the one or more image servers 560 or upload new images to the one or more image servers 560.

The one or more image servers 560 can communicate with the telehealth platform 550 to automatically receive an image and validate the image for a given patient. For example, the image can be added to a patient record as submitted but not validated. This image can be shared with the remote telehealth provider but be flagged as unvalidated. A telehealth visit can be run in parallel with the image validation. In some cases, the patient, using an electronic device, interacts directly with the one or more image servers 560 to upload an image, e.g., from a stored image on the user device or takes a current image using a patient electronic device. In some cases, the patient interacts with the one or more image servers 560 indirectly through an interface of the telehealth platform 550 and can upload multiple images files.

As used herein, the term “client device” may refer to any machine that interfaces to a communications network (such as network 530) to access the telehealth platform 550 and may be the same device as the user device discussed above. The client device 550 may be, but is not limited to, a mobile phone, desktop computer, laptop, portable digital assistants (PDAs), smart phones, a wearable device (e.g., a smart watch), tablets, ultrabooks, netbooks, laptops, multi-processor systems, microprocessor-based or programmable consumer electronics, game consoles, set-top boxes, or any other communication device that a user may use to access a network or the telehealth platform 550. The client device 510, when loaded with software associated with the present disclosure, is a dedicated machine.

In some cases, the telehealth platform 550 is accessible over a global communication system, e.g., the Internet or world wide web. In such instances, the telehealth platform 550 hosts a website (graphical user interface(s) and software to present data and receive input data) that is accessible to the client devices 510. Upon accessing the website, the client devices 510 provide secure login credentials, which are used to access a profile associated with the login credentials and one or more patient profiles or patient information. As used herein, patient information includes any medical information associated with a patient including one or more prior medical insurance claims that were approved or denied, one or more electronic health records or medical health records, patient health information, patient demographic information, prior bloodwork results, prior results of non-bloodwork tests, medical history, medical provider notes in the electronic health record, intake forms completed by the patient, patient in-network insurance coverage, patient out-of-network insurance coverage, patient location, one or more treatment preferences, and/or images. One or more user interfaces associated with the telehealth platform 550 are provided over the Internet via the website to the client devices 510.

Healthcare provider devices 520 can include the same or similar functionality as client devices 510 for accessing the telehealth platform 550 and can be the same device as the aforementioned healthcare provider device. In some cases, the healthcare provider devices 520 are used by “internal” users. Internal users are medical professionals, such as medical personnel, physicians, clinicians, healthcare providers, health-related coaches pharmacy benefit manager (PBM) operators, pharmacists, specialty pharmacy operators or pharmacists, or the like that are associated with, certified by, or employed by one or more organizations that provides the telehealth platform 550. In some cases, the healthcare provider devices 520 are used by “external” users. External users are medical professionals and personnel, such as physicians, clinicians, and health-related coaches that are associated with or employed by a different (external) organization than that which provides the telehealth platform 550.

The healthcare provider devices 520 in some cases are used to access a respective server of the image servers 560. For example, a first group of healthcare provider devices 520 can access a first set of the image servers 560 that provide image processing as described herein. The first group and/or a second group of healthcare provider devices 520 can access a second set of the image servers 560 that provide validation of medical images of a second type or that are affiliated with a second provider or organization.

The healthcare provider devices 520, when used by internal or external users, to access the telehealth platform 550 can view many records, including images, associated with many different patients (or users associated with client devices 510). Different levels of authorization can be associated with different internal and different external users to control, and the healthcare provider devices 520 can control which internal and external users have access to the records. In some instances, only records associated with those patients to which a given internal or external user is referred, are made accessible and available to the given internal or external user device. Sometimes, a first internal or external user can refer a patient or records associated with the patient to a second internal or external user. In such circumstances, the second internal or external user becomes automatically authorized to access and view the patient's records that were referred by the first internal or external user.

The healthcare provider devices 520 can access the telehealth platform 550 in order to review various recommendations that are provided to one or more patients by the image validation system 556 prior to the patient having at an appointment, e.g., a telehealth visit or an in-person visit. Specifically, the image validation system 556 can generate an alert to the healthcare provider devices 520 that identifies a set of image validations that were generated for a given patient. The alert can identify the patient, include various patient profile information that cause the image validations to be generated by the image validation system 556. The medical professional can review the validated image using the healthcare provider devices 520. In some cases, the alert can provide an image that is not validated or flagged as fake to the medical professional via the healthcare provider devices 520.

In some examples, the telehealth platform 550 (and specifically the image validation system 556) can implement a ML technique or ML model, such as a neural network (e.g., as discussed herein). The ML model can be trained to establish a relationship between a plurality of training patient information features and types of images based on visits to various types of medical professionals and associated with a specific diagnosis or suspected diagnosis. The ML model can then receive a new set of patient images of a patient to revise the predictive model that is used to designate submitted images as valid and of sufficient quality for a medical provider to use in a diagnosis or as invalid. The ML model can automatically flag submitted images as fake or of insufficient quality for the provider to use in a patient visit. The present image validation system can request new images from a patient through the patient device while the patient is waiting for connection to a medical provider. In this way, the medical visit can be handled more efficiently by the medical professional as a necessary and quality image associated with the medical issue are available to the medical professional during the telehealth visit. This should increase the efficiency of the telehealth visit and allow for a more accurate diagnosis.

In an example, an app can be downloaded from the telehealth platform 550 to the client device 510 that reviews the image at the client device 510 for minimum requirements for the associated reason for the telehealth visit. If an image taken by the client device 510 does not meet the image control values, the app instructs the patient to take another image with the client device 510. This should decrease the quantity of data being sent to and stored by the image servers 560.

In an example, the ML model can be trained by obtaining a batch of training data comprising a first set of the plurality of training patient image features associated with a first type of image performed based on visits to a first type of medical professional. The ML model processes the first set of the plurality of training patient image features by the ML model to generate a predictive model for validated images for the first type of medical professional. The training data can also include old uploaded images that a medical provider has flagged, using the healthcare provider device 520, as not being valid for use in the diagnosis of a medical issue.

In some examples, the ML model is further trained by obtaining a second batch of training data comprising a second set of the plurality of images associated with a second type of medical issue performed based on visits to a second type of medical professional. The ML model processes the second set of the plurality of images to generate a predictive model for validating images for the second type of medical professional. The training data can also include old uploaded images that the second type of medical professional has flagged, using the healthcare device 520, as not being valid for use in the diagnosis of a medical issue.

In some examples, the ML model is further trained by obtaining a third batch of training data comprising a third set of the plurality of images associated with a second type of medical issue performed based on visits to a third type of medical professional. The ML model processes the second set of the plurality of images to generate a predictive model for validating images for the third type of medical professional. The training data can also include old uploaded images that the third type of medical professional has flagged, using the healthcare device 520, as not being valid for use in the diagnosis of a medical issue.

These training operations can be repeated for multiple batches of training data and/or until a stopping criterion is reached.

In some examples, the image validation system 556 receives a request to analyze the validity of an image. The image validation system 556 accesses patient information associated with a patient and analyzes metadata of the image. Using the metadata, the image validation system is able to determine of the image was generated recently by comparing the present time and day to the time and date that the image was generated to determine if the patient using the client device 510 actually captured the image recently or if the image was captured a long time ago. The image validation system can then reject the image if it is determined that the image was not taken recently. The image validation system 556 may also analyze the data to determine an image capture location. The image validation system 556 can compare the location of the image to the patient's current location, and if there is a substantial discrepancy between these two locations, then the image validation system 556 can reject the image. For example, if the metadata identifies the image as having been captured in a first location and the patient indicates that they are in a very distant second location, then the image validation system 556 can automatically reject the image.

In some examples, the patient information includes at least one information for the patient, patient health information, patient demographic information, prior bloodwork results, prior results of non-bloodwork tests, medical history, medical provider notes in the electronic health record, intake forms completed by the patient, patient in-network insurance coverage, patient out-of-network insurance coverage, patient location, or one or more treatment preferences. In some examples, the image validation system 556 applies the model by: generating a profile of patients associated with the one or more medical tests and determining that one or more attributes of the patient information match the profile of the patients. The model can be applied in response to determining that the one or more attributes of the patient information match the profile of the patient.

In some examples, the image validation system 556 receives feedback from the medical professional during or after the telehealth appointment with the patient. The feedback can be an electronic record that is stored in a memory. The feedback can include a confirmation or rejection of the image. The image validation system 556 can retrain the model by updating one or more parameters of the model based on the feedback provided by the medical professional to the patient to refine future predictions made by the model.

The network 530 may include, or operate in conjunction with, an ad hoc network, an intranet, an extranet, a virtual private network (VPN), a local area network (LAN), a wireless network, a low energy Bluetooth (BLE) connection, a WiFi direct connection, a wireless LAN (WLAN), a wide area network (WAN), a wireless WAN (WWAN), a metropolitan area network (MAN), the Internet, a portion of the Internet, a portion of the Public Switched Telephone Network (PSTN), a plain old telephone service (POTS) network, a cellular telephone network, a wireless network, a Wi-Fi® network, another type of network, or a combination of two or more such networks. For example, a network or a portion of a network may include a wireless or cellular network and the coupling may be a Code Division Multiple Access (CDMA) connection, a Global System for Mobile communications (GSM) connection, or other type of cellular or wireless coupling. In this example, the coupling may implement any of a variety of types of data transfer technology, such as Single Carrier Radio Transmission Technology (1×RTT), Evolution-Data Optimized (EVDO) technology, General Packet Radio Service (GPRS) technology, Enhanced Data rates for GSM Evolution (EDGE) technology, third Generation Partnership Project (3GPP) including 3G, fourth generation wireless (4G) networks, fifth generation wireless (5G) networks, subsequent wireless network protocols, Universal Mobile Telecommunications System (UMTS), High Speed Packet Access (HSPA), Worldwide Interoperability for Microwave Access (WiMAX), Long Term Evolution (LTE) standard, others defined by various standard setting organizations, other long range protocols, or other data transfer technology.

The healthcare provider devices 520 can be used to access pharmacy claims, medical data (e.g., medical information 630 stored in database 552), laboratory data and the like for one or more patients that the healthcare provider devices 520 are authorized to view. This patient information 610 can be maintained in a database 552 by the telehealth platform 550 or in a third-party database accessible to the telehealth platform 550 and/or the healthcare provider devices 520.

In some embodiments, the client devices 510 and the telehealth platform 550 can be communicatively coupled via an audio call (e.g., VOIP, Public Switched Telephone Network, cellular communication network, etc.) or via electronic messages (e.g., online chat, instant messaging, text messaging, email, and the like). While FIG. 5 illustrates a single client device 510 and a single healthcare provider device 520, it is understood that a plurality of such devices can be included in the system 500 in other embodiments. As used herein, the term “client device” may refer to any machine that interfaces to a communications network (such as network 530) to obtain resources from one or more server systems or other client devices.

In some embodiments, the telehealth platform 550 includes the image validation system 556. In some examples, the image validation system 556 processes medical information input by a patient via the client device 510 and/or patient information stored in one or more databases. For example, the client device 510 can present a graphical user interface to the patient. The graphical user interface can receive input from the patient that provides a variety of medical information, such as patient health information, patient demographic information, patient in-network insurance coverage, patient out-of-network insurance coverage, patient location, and/or one or more treatment preferences. The input can also identify storage locations of one or more electronic health records and/or claims information.

In some cases, the patient health information can be input by receiving a selection from the patient of one or more checkboxes from a user interface, each associated with a different medical condition, such as high blood pressure, diabetes, obesity, demographics, and so forth. The patient in-network insurance coverage and out-of-network insurance coverage can also be input by receiving a selection from the patient of one or more checkboxes from a user interface, each associated with a different health insurance carrier. Based on the selected health insurance carrier and health plan information input by the patient, the patient in-network and out-of-network insurance coverages can be automatically determined and retrieved. The patient location can be automatically determined by accessing location information (e.g., GPS coordinates) from the client device 510 and/or by requesting a residential address from the patient. The treatment preferences can be input by receiving selection from the patient specifying how the patient likes to receive treatment, such as in-person, virtually, in a structured format, in aspirational format (e.g., a format that lacks structure), and so forth.

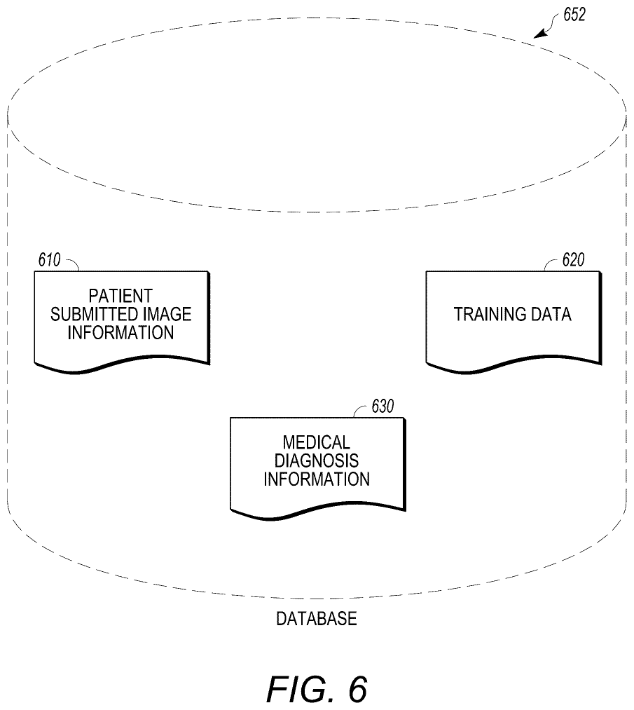

FIG. 6 is an example database 552 that may be deployed within the system of FIG. 5, according to some embodiments. The database 552 includes patient information 610, medical testing information 630, and training data 620. The patient information 610 can be generated or accessed by the telehealth platform 550. For example, the telehealth platform 550 can access the image provided by the patient as well as one or more patient records from one or more sources, including pharmacy claims, benefit information, prescribing physician information, dispensing information (e.g., where and how the patient obtains their current medications), demographic information, prescription information including dose quantity and interval, and input from a patient received via a user interface presented on the client device 510 and so forth. The telehealth platform 550 can collect this information from the patient records and generates a patient features vector that includes this information.

The training data 920 includes a plurality of images saved in a database and past diagnoses from healthcare professionals. The training data 920 is used to train a ML model implemented by the service of image validation system 556 to generate estimates or predictions of whether future images uploaded by patients are of sufficient quality for the healthcare professional to make a diagnosis and also for the ML model to make a diagnosis. Training data 620 can be built over time by identifying a first set of the plurality of training patient information features that are associated with a given diagnosis. The training data 620 can also be built around a large database of patient information, e.g., terabytes. The model also takes into account the past diagnoses based on patients similar to the present patient who have participated in telehealth visits and received diagnoses.

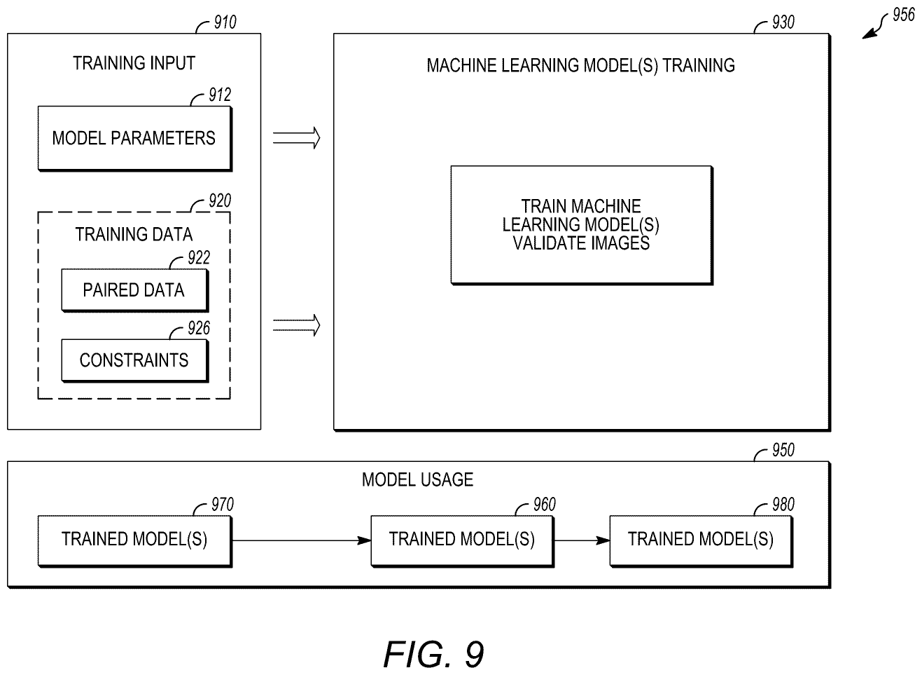

FIG. 9 is a block diagram of an example service of image validation system 556 that may be deployed within the system of FIG. 5, according to some embodiments. Training input 910 includes model parameters 912 and training data 920 (e.g., training data 620 (FIG. 6)) which may include paired training data sets 922 (e.g., input-output training pairs) and constraints 926. Model parameters 912 stores or provides the parameters or coefficients of corresponding ones of ML models. During training, these parameters 912 are adapted based on the input-output training pairs of the training data sets 922. After the parameters 912 are adapted (after training), the parameters are used by trained models 960 to implement the trained ML models on a new set of data 970.

ML model(s) training 930 trains one or more ML techniques based on the sets of input-output pairs of paired training data sets 922. For example, the model training 930 may train the ML model parameters 912 by minimizing a loss function based on one or more ground-truth type of service of care. The ML model can include any one or combination of classifiers or neural networks, such as an artificial neural network, a convolutional neural network, an adversarial network, a generative adversarial network, a deep feed forward network, a radial basis network, a recurrent neural network, a long/short term memory network, a gated recurrent unit, an auto encoder, a variational autoencoder, a denoising autoencoder, a sparse autoencoder, a Markov chain, a Hopfield network, a Boltzmann machine, a restricted Boltzmann machine, a deep belief network, a deep convolutional network, a deconvolutional network, a deep convolutional inverse graphics network, a liquid state machine, an extreme learning machine, an echo state network, a deep residual network, a Kohonen network, a support vector machine, a neural Turing machine, and the like.

Particularly, the ML model can be applied to a training the image validation system to generate more accurate predictions of whether an image is sufficiently clear of an ailment so that a healthcare professional can make a diagnosis and also to generate a more accurate prediction of what diagnosis the healthcare professional will make. The ML model may also take into account the known demographics and health information of the patient requesting the telehealth appointment with the healthcare professional. In some implementations, a derivative of a loss function is computed based on a comparison of the diagnosis predictions with the actual diagnoses by the healthcare professional, and the parameters of the ML model are updated based on the computed derivative of the loss function.

In one example, the ML model receives a batch of training data that includes a first set of the plurality of training images associated with a ground truth first diagnosis. The ML model generates a feature vector based on the first set of the plurality of training images and generates an estimated set of diagnoses. The prediction is compared with the ground truth first type of diagnoses based on actual diagnoses by healthcare professionals and one or more parameters of the ML model are updated based on the comparison.

The result of minimizing the loss function for multiple sets of training data trains, adapts, or optimizes the model parameters 912 of the corresponding ML models. In this way, the ML model is trained to establish a relationship between a plurality of training images and diagnoses by healthcare professionals.

For example, the ML model receives a second batch of training data comprising a second set of the plurality of training images associated with a ground truth second type of diagnoses by healthcare professionals. The ML model generates a feature vector based on the second set of the plurality of training images and generates an estimated second estimated set of diagnoses prior to another given diagnoses by a second type of medical professional. The prediction is compared with the ground truth second type diagnoses by the healthcare professional and one or more parameters of the ML model are updated based on the comparison.

As a further example, the ML model receives a third batch of training data comprising a third set of the plurality of training images associated with a ground truth third type of diagnoses based on diagnoses by healthcare professionals. The ML model generates a feature vector based on the third set of the plurality of training images and generates a third estimated set of predicted diagnoses. The predictions are compared with the ground truth third types of predictions and one or more parameters of the ML model are updated based on the comparison.

After the ML model is trained, new data 970, including one or more images are received. The trained ML technique may be applied to the new data 970 to generate results 980 including predictions on if the images are sufficiently clear to make a diagnoses and also including predictions of the diagnoses. The recommendation made by the image validation system 556 can be represented in a graphical user interface that depicts each of a plurality of different types of medical testing to obtain.

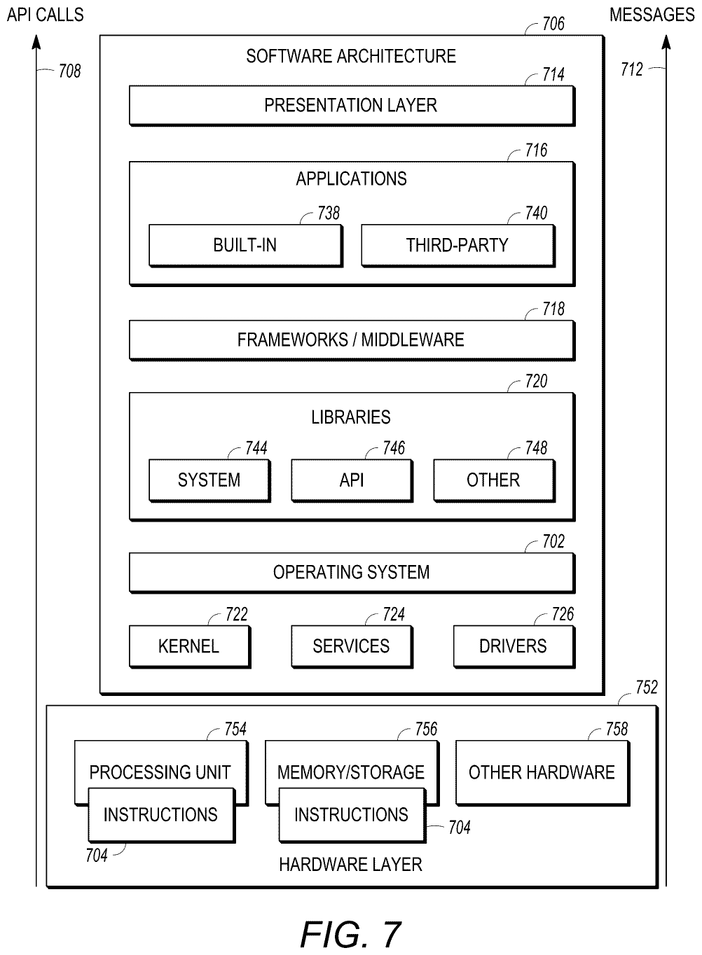

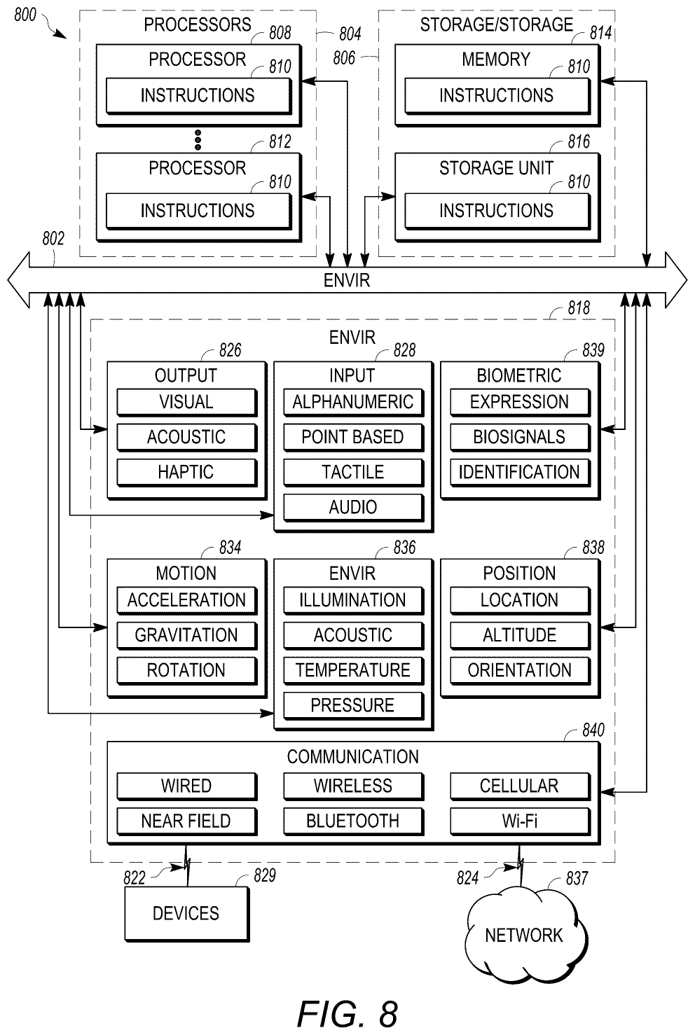

FIG. 7 is a block diagram illustrating an example software architecture 706, which may be used in conjunction with various hardware architectures herein described. FIG. 7 is a non-limiting example of a software architecture and it will be appreciated that many other architectures may be implemented to facilitate the functionality described herein. The software architecture 706 may execute on hardware such as machine 800 of FIG. 8 that includes, among other things, processors 804, memory 814, and input/output (I/O) components 818. A representative hardware layer 752 is illustrated and can represent, for example, the machine 800 of FIG. 8. The representative hardware layer 752 includes a processing unit 754 having associated executable instructions 704. Executable instructions 704 represent the executable instructions of the software architecture 706, including implementation of the methods, components, and so forth described herein. The hardware layer 752 also includes memory and/or storage devices memory/storage 756, which also have executable instructions 704. The hardware layer 752 may also comprise other hardware 758. The software architecture 706 may be deployed in any one or more of the components shown in FIG. 5. The software architecture 706 can be utilized to apply a ML technique or model to generate a prediction of a medical tests to recommend to a patient prior to a scheduled visit to a medical professional.

In the example architecture of FIG. 7, the software architecture 706 may be conceptualized as a stack of layers where each layer provides particular functionality. For example, the software architecture 706 may include layers such as an operating system 702, libraries 720, frameworks/middleware 718, applications 716, and a presentation layer 714. Operationally, the applications 716 and/or other components within the layers may invoke API calls 708 through the software stack and receive messages 712 in response to the API calls 708. The layers illustrated are representative in nature and not all software architectures have all layers. For example, some mobile or special purpose operating systems may not provide a frameworks/middleware 718, while others may provide such a layer. Other software architectures may include additional or different layers.

The operating system 702 may manage hardware resources and provide common services. The operating system 702 may include, for example, a kernel 722, services 724, and drivers 726. The kernel 722 may act as an abstraction layer between the hardware and the other software layers. For example, the kernel 722 may be responsible for memory management, processor management (e.g., scheduling), component management, networking, security settings, and so on. The services 724 may provide other common services for the other software layers. The drivers 726 are responsible for controlling or interfacing with the underlying hardware. For instance, the drivers 726 include display drivers, camera drivers, Bluetooth® drivers, flash memory drivers, serial communication drivers (e.g., Universal Serial Bus (USB) drivers), Wi-Fi® drivers, audio drivers, power management drivers, and so forth depending on the hardware configuration.

The libraries 720 provide a common infrastructure that is used by the applications 716 and/or other components and/or layers. The libraries 720 provide functionality that allows other software components to perform tasks in an easier fashion than to interface directly with the underlying operating system 702 functionality (e.g., kernel 722, services 724 and/or drivers 726). The libraries 720 may include system libraries 744 (e.g., C standard library) that may provide functions such as memory allocation functions, string manipulation functions, mathematical functions, and the like. In addition, the libraries 720 may include API libraries 746 such as media libraries (e.g., libraries to support presentation and manipulation of various media format such as MPREG4, H.264, MP3, AAC, AMR, JPG, PNG), graphics libraries (e.g., an OpenGL framework that may be used to render two-dimensional and three-dimensional in a graphic content on a display), database libraries (e.g., SQLite that may provide various relational database functions), web libraries (e.g., WebKit that may provide web browsing functionality), and the like. The libraries 720 may also include a wide variety of other libraries 748 to provide many other APIs to the applications 716 and other software components/devices.

The frameworks/middleware 718 (also sometimes referred to as middleware) provide a higher-level common infrastructure that may be used by the applications 716 and/or other software components/devices. For example, the frameworks/middleware 718 may provide various graphic user interface functions, high-level resource management, high-level location services, and so forth. The frameworks/middleware 718 may provide a broad spectrum of other APIs that may be utilized by the applications 716 and/or other software components/devices, some of which may be specific to a particular operating system 702 or platform.

The applications 716 include built-in applications 738 and/or third-party applications 740. Examples of representative built-in applications 738 may include, but are not limited to, a contacts application, a browser application, a book reader application, a location application, a media application, a messaging application, and/or a game application. Third-party applications 740 may include an application developed using the ANDROID™ or IOS™ software development kit (SDK) by an entity other than the vendor of the particular platform, and may be mobile software running on a mobile operating system such as IOS™, ANDROID™, WINDOWS® Phone, or other mobile operating systems. The third-party applications 740 may invoke the API calls 708 provided by the mobile operating system (such as operating system 702) to facilitate functionality described herein.

The applications 716 may use built-in operating system functions (e.g., kernel 722, services 724, and/or drivers 726), libraries 720, and frameworks/middleware 718 to create UIs to interact with users of the system. Alternatively, or additionally, in some systems, interactions with a user may occur through a presentation layer, such as presentation layer 714. In these systems, the application/component “logic” can be separated from the aspects of the application/component that interact with a user.

FIG. 8 is a block diagram illustrating components of a machine 800, according to some example embodiments, able to read instructions from a machine-readable medium (e.g., a machine-readable storage medium) and perform any one or more of the methodologies discussed herein. Specifically, FIG. 8 shows a diagrammatic representation of the machine 800 in the example form of a computer system, within which instructions 810 (e.g., software, a program, an application, an applet, an app, or other executable code) for causing the machine 800 to perform any one or more of the methodologies discussed herein may be executed. For example, the instructions 810 may be executed by the system 100 to process a combination of patient information features with a trained ML model to predict or condition triggering recommendations for one or more medical tests to the patient associated with the patient information features prior to an upcoming scheduled visit to a medical professional.

As such, the instructions 810 may be used to implement devices or components described herein. The instructions 810 transform the general, non-programmed machine 800 into a particular machine 800 programmed to carry out the described and illustrated functions in the manner described. In alternative embodiments, the machine 800 operates as a standalone device or may be coupled (e.g., networked) to other machines. In a networked deployment, the machine 800 may operate in the capacity of a server machine or a client machine in a server-client network environment, or as a peer machine in a peer-to-peer (or distributed) network environment. The machine 800 may comprise, but not be limited to a server computer, a client computer, a personal computer (PC), a tablet computer, a laptop computer, a netbook, a STB, a PDA, an entertainment media system, a cellular telephone, a smart phone, a mobile device, a wearable device (e.g., a smart watch), a smart home device (e.g., a smart appliance), other smart devices, a web appliance, a network router, a network switch, a network bridge, or any machine capable of executing the instructions 810, sequentially or otherwise, that specify actions to be taken by machine 800. Further, while only a single machine 800 is illustrated, the term “machine” shall also be taken to include a collection of machines that individually or jointly execute the instructions 810 to perform any one or more of the methodologies discussed herein. The machine 800 can send and receive carrier signals that include representations of data on which the machine can operate.

The machine 800 may include processors 804, memory/storage 806, and I/O components 818, which may be configured to communicate with each other such as via a bus 802. In an example embodiment, the processors 804 (e.g., a central processing unit (CPU), a reduced instruction set computing (RISC) processor, a complex instruction set computing (CISC) processor, a graphics processing unit (GPU), a digital signal processor (DSP), an application-specific integrated circuit (ASIC), a radio-frequency integrated circuit (RFIC), another processor, or any suitable combination thereof) may include, for example, a processor 808 and a processor 812 that may execute the instructions 810. The term “processor” is intended to include multi-core processors 804 that may comprise two or more independent processors (sometimes referred to as “cores”) that may execute instructions contemporaneously. Although FIG. 8 shows multiple processors 804, the machine 800 may include a single processor with a single core, a single processor with multiple cores (e.g., a multi-core processor), multiple processors with a single core, multiple processors with multiple cores, or any combination thereof.

The memory/storage 806 may include a memory 814, such as a main memory, or other memory storage, database 152, and a storage unit 816, both accessible to the processors 804 such as via the bus 802. The bus 802 can carry electrical signals, digital or analog, including carrier signals. The storage unit 816 and memory 814 store the instructions 810 embodying any one or more of the methodologies or functions described herein. The instructions 810 may also reside, completely or partially, within the memory 814, within the storage unit 816, within at least one of the processors 804 (e.g., within the processor's cache memory), or any suitable combination thereof, during execution thereof by the machine 800. Accordingly, the memory 814, the storage unit 816, and the memory of processors 804 are examples of machine-readable media.

The I/O components 818 may include a wide variety of components to receive input, provide output, produce output, transmit information, exchange information, capture measurements, and so on. The specific I/O components 818 that are included in a particular machine 800 will depend on the type of machine. For example, portable machines such as mobile phones will likely include a touch input device or other such input mechanisms, while a headless server machine will likely not include such a touch input device. It will be appreciated that the I/O components 818 may include many other components that are not shown in FIG. 8. The I/O components 818 are grouped according to functionality merely for simplifying the following discussion and the grouping is in no way limiting. In various example embodiments, the I/O components 818 may include output components 826 and input components 828. The output components 826 may include visual components (e.g., a display such as a plasma display panel (PDP), a light emitting diode (LED) display, a liquid crystal display (LCD), a projector, or a cathode ray tube (CRT)), acoustic components (e.g., speakers), haptic components (e.g., a vibratory motor, resistance mechanisms), other signal generators, and so forth. The input components 828 may include alphanumeric input components (e.g., a keyboard, a touch screen configured to receive alphanumeric input, a photo-optical keyboard, or other alphanumeric input components), point-based input components (e.g., a mouse, a touchpad, a trackball, a joystick, a motion sensor, or other pointing instrument), tactile input components (e.g., a physical button, a touch screen that provides location and/or force of touches or touch gestures, or other tactile input components), audio input components (e.g., a microphone), and the like.

In further example embodiments, the I/O components 818 may include biometric components 839, motion components 834, environmental components 836, or position components 838 among a wide array of other components. For example, the biometric components 839 may include components to detect expressions (e.g., hand expressions, facial expressions, vocal expressions, body gestures, or eye tracking), measure biosignals (e.g., blood pressure, heart rate, body temperature, perspiration, or brain waves), identify a person (e.g., voice identification, retinal identification, facial identification, fingerprint identification, or electroencephalogram based identification), and the like. The motion components 834 may include acceleration sensor components (e.g., accelerometer), gravitation sensor components, rotation sensor components (e.g., gyroscope), and so forth. The environmental components 836 may include, for example, illumination sensor components (e.g., photometer), temperature sensor components (e.g., one or more thermometer that detect ambient temperature), humidity sensor components, pressure sensor components (e.g., barometer), acoustic sensor components (e.g., one or more microphones that detect background noise), proximity sensor components (e.g., infrared sensors that detect nearby objects), gas sensors (e.g., gas detection sensors to detection concentrations of hazardous gases for safety or to measure pollutants in the atmosphere), or other components that may provide indications, measurements, or signals corresponding to a surrounding physical environment. The position components 838 may include location sensor components (e.g., a GPS receiver component), altitude sensor components (e.g., altimeters or barometers that detect air pressure from which altitude may be derived), orientation sensor components (e.g., magnetometers), and the like.

Communication may be implemented using a wide variety of technologies. The I/O components 818 may include communication components 840 operable to couple the machine 800 to a network 837 or devices 829 via coupling 824 and coupling 822, respectively. For example, the communication components 840 may include a network interface component or other suitable device to interface with the network 837. In further examples, communication components 840 may include wired communication components, wireless communication components, cellular communication components, Near Field Communication (NFC) components, Bluetooth® components (e.g., Bluetooth® Low Energy), Wi-Fi® components, and other communication components to provide communication via other modalities. The devices 829 may be another machine or any of a wide variety of peripheral devices (e.g., a peripheral device coupled via a USB).

Moreover, the communication components 840 may detect identifiers or include components operable to detect identifiers. For example, the communication components 840 may include Radio Frequency Identification (RFID) tag reader components, NFC smart tag detection components, optical reader components (e.g., an optical sensor to detect one-dimensional bar codes such as Universal Product Code (UPC) bar code, multi-dimensional bar codes such as Quick Response (QR) code, Aztec code, Data Matrix, Dataglyph, MaxiCode, PDF417, Ultra Code, UCC RSS-2D bar code, and other optical codes), or acoustic detection components (e.g., microphones to identify tagged audio signals). In addition, a variety of information may be derived via the communication components 840, such as location via Internet Protocol (IP) geo-location, location via Wi-Fi® signal triangulation, location via detecting a NFC beacon signal that may indicate a particular location, and so forth.

Training data 920 includes constraints 926 which may define the constraints of a given image. The paired training data sets 922 may include sets of input-output pairs, such as pairs of a plurality of training images and types of medical tests performed based on visits to various types of medical professionals associated with the training patient information features. Some components of training input 910 may be stored separately at a different off-site facility or facilities than other components.

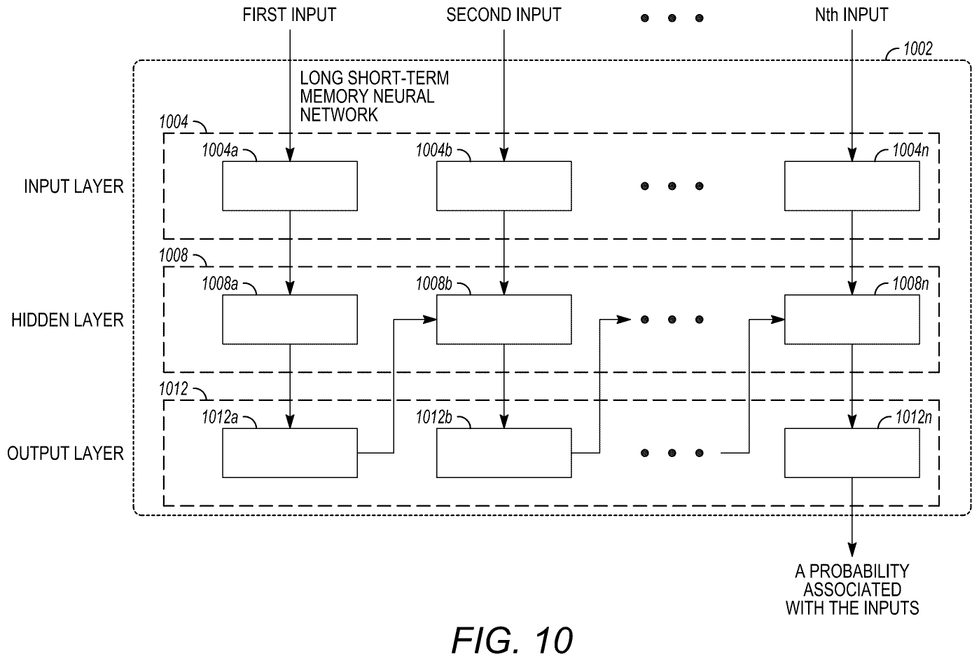

FIG. 10 is a functional block diagram of an example neural network 1002 that can be used for the inference engine or other functions (e.g., engines) as described herein to produce a predictive model. The predictive model can identify if an image submitted by a customer is sufficient for a medical professional to make a diagnosis of an illness and also can make a prediction of the diagnosis itself. In an example, the neural network 1002 can be a LSTM neural network. In an example, the neural network 1002 can be a recurrent neural network (RNN). The example neural network 1002 may be used to implement the ML as described herein, and various implementations may use other types of ML networks. The neural network 1002 includes an input layer 1004, a hidden layer 1008, and an output layer 1012. The input layer 1004 includes inputs 1004a, 1004b . . . 1004n. The hidden layer 1008 includes neurons 1008a, 1008b . . . 1008n. The output layer 1012 includes outputs 1012a, 1012b . . . 1012n.

Each neuron of the hidden layer 1008 receives an input from the input layer 1004 and outputs a value to the corresponding output in the output layer 1012. For example, the neuron 1008a receives an input from the input 1004a and outputs a value to the output 1012a. Each neuron, other than the neuron 1008a, also receives an output of a previous neuron as an input. For example, the neuron 1008b receives inputs from the input 1004b and the output 1012a. In this way the output of each neuron is fed forward to the next neuron in the hidden layer 1008. The last output 1012n in the output layer 1012 outputs a probability associated with the inputs 1004a-1004n. Although the input layer 1004, the hidden layer 1008, and the output layer 1012 are depicted as each including three elements, each layer may contain any number of elements. Neurons can include one or more adjustable parameters, weights, rules, criteria, or the like.

In various implementations, each layer of the neural network 1002 must include the same number of elements as each of the other layers of the neural network 1002. For example, training patient information data features may be processed to create the inputs 1004a-1004n. The neural network 1002 may implement a model to produce one or more medical test recommendations for at least one of the patient information data features and/or a reason for an upcoming scheduled visit to a medical professional of a particular medical professional type. More specifically, the inputs 1004a-1004n can include patient information data features (binary, vectors, factors or the like) stored in the storage device 510. The patient information data features can specify at least one of or combination of a reason for an upcoming visit, past medical professional recommendations, past treatment recommendations, electronic health record, past claims information for the patient, patient health information, patient demographic information, prior bloodwork results, prior results of non-bloodwork tests, medical history, medical provider notes in the electronic health record, intake forms completed by the patient, patient in-network insurance coverage, patient out-of-network insurance coverage, patient location, and/or one or more treatment preferences.

The patient information data features can be provided to neurons 1008a-1008n for analysis and connections between the known facts. The neurons 1008a-1008n, upon finding connections, provides the potential connections as outputs to the output layer 1012, which determines a list of medical tests to perform or obtain from many different types of medical tests.

The neural network 1002 can perform any of the above calculations. The output of the neural network 1002 can be used to trigger service of care type selection to recommend to a patient in a graphical user interface. For example, the notification can be provided to a PBM, health plan manager, pharmacy, physician, caregiver, and/or a patient.

In some embodiments, a convolutional neural network may be implemented. Similar to neural networks, convolutional neural networks include an input layer, a hidden layer, and an output layer. However, in a convolutional neural network, the output layer includes one fewer output than the number of neurons in the hidden layer and each neuron is connected to each output. Additionally, each input in the input layer is connected to each neuron in the hidden layer. In other words, input 1004a is connected to each of neurons 1008a, 1008b . . . 1008n.

The neural network 1002 can use some components of the system 800 to execute the methodology as described herein.

The predictive model network 1002 can further operate to map care plans to identified groups of patient data. That is, the predictive model network 1002 can act to appropriately group certain patient records, which can represent one or more patients. The patient records can include data fields that may be predictive of certain care plans. The care plans can include the schedule of certain medical tests, procedures and review for a given medical state of the grouped patients. One of the care plan steps can be scheduling medical tests and/or procedures prior to an in-person visit to a medical provider, e.g., prior to an office visit with a doctor or specialist.

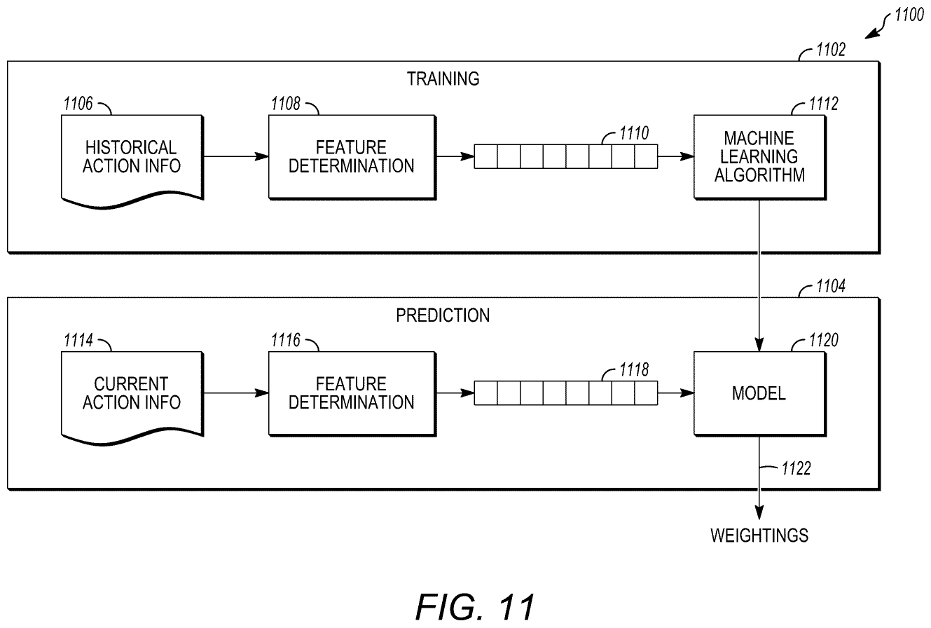

FIG. 11 illustrates a ML engine 1100 for generating a predictive model for approving an image or using an approved image to generate a probable diagnosis to share to the provider via the provider device. The suggest diagnosis can include a suggested prescription. The suggested diagnosis is not shared with the patient or the patient's device. Further, the suggested diagnosis from the predictive model is not entered into the patient's electronic medical records in the database. When a telehealth visit ends, the suggested diagnosis is not associated with the patient. The act that the diagnosis was accurate and approved by the provider device or not approved by the patient device is recorded and fed back into the ML engine to improve the predictive model. The ML engine may be employed within the telehealth system as described herein. A system may calculate one or more weightings for criteria based upon one or more ML algorithms. FIG. 11 shows an example ML engine 1100 according to some examples of the present disclosure.

ML engine 1100 utilizes a training engine 1102 and a prediction engine 1104. Training engine 1102 inputs historical information 1106 for approved images into feature determination engine 1108. Other historical information 1106 may include historical visit records, historical claim records, and other image data that are representative of a disease state. The historical action information 1106 may be labeled with an indication, such as a degree of accuracy of an approved image used in a telehealth visit. The degree of accuracy can be dependent on focus, lighting and contrast in the image. In some examples, an outcome may be subjectively assigned to historical data, but in other examples, one or more labelling criteria may be utilized that may focus on objective outcome metrics, e.g., accuracy of diagnosis by the medical provider.

The above discussion is meant to be illustrative of the principles and various embodiments of the present invention. Numerous variations and modifications will become apparent to those skilled in the art once the above disclosure is fully appreciated. It is intended that the following claims be interpreted to embrace all such variations and modifications.

While many of the described embodiments are directed toward verifying that uploaded images from a patient are of sufficient quality to aide a healthcare provider, over a remote, non-inperson healthcare visit, in diagnosing a patient, the present image system can also be used to verify the identity of a patient. Along with illness images, the present system can develop a (AI/ML) predictive model to identify the patient based on prior images of the patient and a current image. The current image can be a frame capture of the video stream from the patient device or be an uploaded image of the patient's choosing. Thus, the present disclosure includes a first image approval of the patient and a second image approval relating to the health condition of the patient.

The foregoing description is merely illustrative in nature and is in no way intended to limit the disclosure, its application, or uses. The broad teachings of the disclosure can be implemented in a variety of forms. Therefore, while this disclosure includes particular examples, the true scope of the disclosure should not be so limited since other modifications will become apparent upon a study of the drawings, the specification, and the following claims. It should be understood that one or more steps within a method may be executed in different order (or concurrently) without altering the principles of the present disclosure. Further, although each of the embodiments is described above as having certain features, any one or more of those features described with respect to any embodiment of the disclosure can be implemented in and/or combined with features of any of the other embodiments, even if that combination is not explicitly described. In other words, the described embodiments are not mutually exclusive, and permutations of one or more embodiments with one another remain within the scope of this disclosure.

Spatial and functional relationships between elements (for example, between modules) are described using various terms, including “connected,” “engaged,” “interfaced,” and “coupled.” Unless explicitly described as being “direct,” when a relationship between first and second elements is described in the above disclosure, that relationship encompasses a direct relationship where no other intervening elements are present between the first and second elements, and also an indirect relationship where one or more intervening elements are present (either spatially or functionally) between the first and second elements. As used herein, the phrase at least one of A, B, and C should be construed to mean a logical (A OR B OR C), using a non-exclusive logical OR, and should not be construed to mean “at least one of A, at least one of B, and at least one of C.”

In the figures, the direction of an arrow, as indicated by the arrowhead, generally demonstrates the flow of information (such as data or instructions) that is of interest to the illustration. For example, when element A and element B exchange a variety of information but information transmitted from element A to element B is relevant to the illustration, the arrow may point from element A to element B. This unidirectional arrow does not imply that no other information is transmitted from element B to element A. Further, for information sent from element A to element B, element B may send requests for, or receipt acknowledgements of, the information to element A. The term subset does not necessarily require a proper subset. In other words, a first subset of a first set may be coextensive with (equal to) the first set.

In this application, including the definitions below, the term “module” or the term “controller” may be replaced with the term “circuit.” The term “module” may refer to, be part of, or include processor hardware (shared, dedicated, or group) that executes code and memory hardware (shared, dedicated, or group) that stores code executed by the processor hardware.

The module may include one or more interface circuits and circuitry. In some examples, the interface circuit(s) may implement wired or wireless interfaces that connect to a local area network (LAN) or a wireless personal area network (WPAN). Examples of a LAN are Institute of Electrical and Electronics Engineers (IEEE) Standard 802.11-2016 (also known as the WIFI wireless networking standard) and IEEE Standard 802.3-2015 (also known as the ETHERNET wired networking standard). Examples of a WPAN are the BLUETOOTH wireless networking standard from the Bluetooth Special Interest Group and IEEE Standard 802.15.4.

The module may communicate with other modules using the interface circuit(s). Although the module may be depicted in the present disclosure as logically communicating directly with other modules, in various implementations the module may actually communicate via a communications system. The communications system includes physical and/or virtual networking equipment such as hubs, switches, routers, and gateways. In some implementations, the communications system connects to or traverses a wide area network (WAN) such as the Internet. For example, the communications system may include multiple LANs connected to each other over the Internet or point-to-point leased lines using technologies including Multiprotocol Label Switching (MPLS) and virtual private networks (VPNs).

In various implementations, the functionality of the module may be distributed among multiple modules that are connected via the communications system. For example, multiple modules may implement the same functionality distributed by a load balancing system. In a further example, the functionality of the module may be split between a server (also known as remote, or cloud) module and a client (or, user) module.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of a non-transitory computer-readable medium are nonvolatile memory devices (such as a flash memory device, an erasable programmable read-only memory device, or a mask read-only memory device), volatile memory devices (such as a static random access memory device or a dynamic random access memory device), magnetic storage media (such as an analog or digital magnetic tape or a hard disk drive), and optical storage media (such as a CD, a DVD, or a Blu-ray Disc).

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

The computer programs include processor-executable instructions that are stored on at least one non-transitory computer-readable medium. The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc.

The computer programs may include: (i) descriptive text to be parsed, such as HTML (hypertext markup language), XML (extensible markup language), or JSON (JavaScript Object Notation), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Swift, Haskell, Go, SQL, R, Lisp, Java®, Fortran, Perl, Pascal, Curl, OCaml, Javascript®, HTML5 (Hypertext Markup Language 5th revision), Ada, ASP (Active Server Pages), PHP (PHP: Hypertext Preprocessor), Scala, Eiffel, Smalltalk, Erlang, Ruby, Flash®, Visual Basic®, Lua, MATLAB, SIMULINK, and Python®.

A mainframe can be the central computing server in a client/server system. The mainframe can have high processing power, memory and storage to support massive data processing operations. In contrast, cloud computing is a distributed architecture of multiple systems (e.g., servers smaller than a mainframe) that may communicate with each other over a network, e.g., Internet, web or other communication channels and standards. In some examples, cloud computing is on-demand and can share hardware resources with other users. This can lead to lower costs of use compared to mainframes which require the ownership of sufficient hardware to exceed the future maximum data processing requirements.

Implementations of the systems, algorithms, methods, instructions, etc., described herein may be realized in hardware, software, or any combination thereof. The hardware may include, for example, computers, intellectual property (IP) cores, application-specific integrated circuits (ASICs), programmable logic arrays, optical processors, programmable logic controllers, microcode, microcontrollers, servers, microprocessors, digital signal processors, or any other suitable circuit. In the claims, the term “processor” should be understood as encompassing any of the foregoing hardware, either singly or in combination. The terms “signal” and “data” are used interchangeably.

What is claimed is:

1. A system for providing telehealth services to a patient, the system comprising:
a computing device that includes at least one processor and at least one memory including instructions that, when executed by the at least one processor, cause the at least one processor to:
receive inputs from a patient device, the inputs including at least health data of the patient and an image of an ailment;
with an image prediction machine learning model, determine if the image is of sufficient quality to generate one or more predictions of the ailment;
with an ailment prediction machine learning model, generate one or more predictions of the ailment based on the health data of the patient and the image of the ailment;
transmit the one or more predictions of the ailment and the image to a healthcare provider device;
establish communication between the patient device and the healthcare provider device;
receive inputs from the healthcare provider device, the inputs from the healthcare provider device including at least a confirmation or a rejection of the image and including at least one or more predictions of the ailment; and
train, based on the inputs from the healthcare provider, the image prediction machine learning model and the ailment prediction machine learning model.

2. The system as set forth in claim 1, wherein the at least one memory further includes instructions that, when executed by the at least one processor, cause the processor to:
with the ailment prediction machine learning model, generate one or more predictions of a treatment plan for the patient;
transmit the one or more predictions of the treatment plan for the patient to the healthcare provider device; and
wherein the inputs from the healthcare provider device include a confirmation or rejection of the one or more predictions of the treatment plan for the patient.

3. The system as set forth in claim 1, further including a database that retains images for comparison by the image prediction machine learning model and the ailment prediction machine learning model.

4. The system as set forth in claim 1, wherein the inputs from the healthcare provider device include a confirmation or a rejection of the image.

5. The system as set forth in claim 1, wherein the at least one memory further includes instructions that, when executed by the at least one processor, cause the processor to:
with the image prediction machine learning model, determine if the image of the ailment is a false image that was not taken by the patient; and
in response to a determination that the image is a false image, request an additional image from the patient device.

6. The system as set forth in claim 1, wherein the at least one memory further includes instructions that, when executed by the at least one processor, cause the processor to:
select the healthcare provider device to transmit the one or more predictions of the ailment to among a plurality of healthcare provider devices based on the one or more predictions of the ailment.

7. The system as set forth in claim 1, wherein the at least one memory further includes instructions that, when executed by the at least one processor, cause the processor to:
enhance the image prior to transmitting the image to the healthcare provider device.

8. The system as set forth in claim 1, wherein the computing device is a server that is remote from the patient device and from the healthcare provider device.

9. The system as set forth in claim 1, wherein the computing device is associated with the healthcare provider device.

10. A method performed by a system for providing telehealth services to a patient, the system comprising a computing device with at least one processor and at least one memory, the method comprising the steps of:
receiving inputs from a patient device, the inputs including at least health data of the patient and an image of an ailment;
with an image prediction machine learning model, determining if the image is of sufficient quality to generate one or more predictions of the ailment;
with an ailment prediction machine learning model, generating one or more predictions of the ailment based on the health data of the patient and the image of the ailment;
transmitting the one or more predictions of the ailment and the image to a healthcare provider device;
establishing communication between the patient device and the healthcare provider device;
receiving inputs from the healthcare provider device, the inputs including at least a confirmation or a rejection of the image and including a confirmation or a rejection of the one or more predictions of the ailment; and
training, based on the inputs from the healthcare provider, the image prediction machine learning model and the ailment prediction machine learning model.

11. The method as set forth in claim 10, wherein the method further includes the steps of:
with the ailment prediction machine learning model, generating one or more predictions of a treatment plan for the patient;
transmitting the one or more predictions of the treatment plan for the patient to the healthcare provider device; and
wherein the inputs from the healthcare provider device include a confirmation or rejection of the one or more predictions of the treatment plan for the patient.

12. The method as set forth in claim 10, further including the step of storing the image in a database for access by the image prediction machine learning model and the ailment prediction machine learning model.

13. The method as set forth in claim 10, wherein the inputs from the healthcare provider device include a confirmation or a rejection of the image.

14. The method as set forth in claim 10, further including the steps of:
with the image prediction machine learning model, determining if the image of the ailment is a false image that was not taken by the patient; and
in response to a determination that the image is a false image, requesting an additional image from the patient device.

15. The method as set forth in claim 10, further including the step of:
selecting the healthcare provider device to transmit the one or more predictions of the ailment to among a plurality of healthcare provider devices based on the one or more predictions of the ailment.

16. The method as set forth in claim 10, further including the step of:
enhancing the image prior to transmitting the image to the healthcare provider device.

17. A system for providing telehealth services to a patient, comprising:
a cloud computing device that includes at least one processor and at least one memory, the memory including instructions that, when executed by the processor, cause the processor to:
receive inputs from a patient device, the inputs including at least health data of the patient and an image of an ailment;
with an image prediction machine learning model, determine if the image is of sufficient quality to generate one or more predictions of the ailment;
with an ailment prediction machine learning model, generate one or more predictions of the ailment and one or more predictions of a treatment plan based on the health data of the patient and the image of the ailment;
select a healthcare provider device of a plurality of healthcare provider devices based on the one or more predictions of the ailment;
transmit the one or more predictions of the ailment and the one or more predictions of the treatment plan and the image to the healthcare provider device;
establish communication between the patient device and the healthcare provider device;
receive inputs from the healthcare provider device, the inputs from the healthcare provider device including at least a confirmation or rejection of the image, a confirmation or a rejection of the one or more predictions of the ailment, and a confirmation or rejection of the one or more predictions of the treatment plan; and
train, based on the inputs from the healthcare provider, the image prediction machine learning model and the ailment prediction machine learning model.

18. The system as set forth in claim 17, wherein the at least one memory further includes instructions that, when executed by the at least one processor, cause the processor to:
in response to a determination that the image of the ailment is not of sufficient quality to generate the one or more predictions of the ailment, request an additional image from the patient device.

19. The system as set forth in claim 18, wherein the inputs from the healthcare provider device include a confirmation or a rejection of the image.

20. The system as set forth in claim 18, wherein the at least one memory further includes instructions that, when executed by the at least one processor, cause the processor to:
with the image prediction machine learning model, determine if the image of the ailment is a false image that was not taken by the patient; and
in response to a determination that the image is a false image, request an additional image from the patient device.
